(19) United States
(12) Patent Application Publication
(10) Pub. No.: US 2007/0281288 A1
(43) Pub. Date: Dec. 6, 2007

(54) Method and System for Detecting Analytes

(76) Inventors: Shimshon Belkin, Kiryat Ono (IL); Rami Pedahzur, Doar-Na Harei Yehuda (IL); Rachel Rosen, Modiin (IL); Itai Benovici, Jerusalem (IL); Yosi Shacham-Diamand, Zikhron-Yaakov (IL); Arthur Rabner, Natania (IL); Mark Oksman, Bat-Yam (IL)

Correspondence Address: Martin D. Moynihan;PRTSI, P.O.Box 16446, Arlington, VA, 22215, US

(21) Appl. No.: 10/587,516

(22) PCT Filed: Jan. 25, 2005
(86) PCT No.: PCT/IL05/00090

§ 371 Date: Jun. 6, 2007

Related U.S. Application Data
(60) Provisional application No. 60/539,097, filed on Jan. 27, 2004.
(60) Provisional application No. 60/565,850, filed on Apr. 28, 2004.

(51) Int. Cl. C12M 1/34; C12Q 1/00; G01J 1/58

(52) U.S. Cl. 435/4; 250/458.1; 435/287.1; 435/288.7

(57) ABSTRACT

A device for detecting a presence of an analyte in a sample. The device comprises a device body configured with at least one reaction chamber configured for containing a sensor capable of producing a detectable signal when exposed to the analyte in the sample. The reaction chambers are in fluid communication with at least one sample port and at least one actuator port via a first set of microfluidic channels arranged such that application of a negative pressure to actuator port delivers fluid placed in the sample port to the reaction chambers.

### FIELD AND BACKGROUND OF THE INVENTION

The present invention relates to a method and system for detecting and/or identifying analytes. In particular, the present invention relates to a device utilizing electro mechanical technology for the purpose of, for example, determining toxicity of a fluid.

Much industrial and academic effort is presently directed at the development of integrated micro devices or systems combining electrical, mechanical and/or optical/electrooptical components, commonly known as Micro Electro Mechanical Systems (MEMS). MEMS are fabricated using integrated circuit batch processing techniques and can range in size from micrometers to millimeters. These systems can sense, control and actuate on the micro scale, and function individually or in arrays to generate effects on the macro scale.

The development of miniaturized devices for chemical analysis and for synthesis and fluid manipulation is motivated by the prospects of improved efficiency, reduced cost and enhanced accuracy. Efficient, reliable manufacturing processes are a critical requirement for the cost-effective, high-volume production of devices that are targeted at high-volume, high-throughput test markets.

In the most general form, MEMS consist of mechanical microstructures, microsensors, microactuators and electronics which are integrated into a single device or platform (e.g., on a silicon chip). The microfabrication technology enables fabrication of large arrays of devices, which individually perform simple tasks but in combination can accomplish complicated functions.

One type of MEMS is a microfluidic device. Microfluidic devices include components such as channels, reservoirs, mixers, pumps, valves, chambers, cavities, reaction chambers, heaters, fluidic interconnects, diffusers, nozzles, and other microfluidic components. These microfluidic components typically have dimensions which range between several micrometers to several millimeters. The small dimensions of such components minimize the physical size, the power consumption, the response time and the waste of a microfluidic device as compared to other technologies.

In the area of life science, microfluidic devices are used in DNA chips, protein chips and total analysis systems (also known as lab-on-chip). The use of a microfluidic device in the fabrication process of a microchip facilitates the production of small and high-density spots on the substrate. Since only a small amount of solution is needed to make one chip, the cost of chip production is substantially reduced. In addition, a microfluidic device can created spots in consistent quantities and with uniform configurations, so as to enable highly accurate comparisons between spots.

Microfluidic devices are typically used in genetic, chemical, biochemical, pharmaceutical, biomedical, chromatography, medical, radiological and environmental applications. For example, in environmental applications, such devices are used for detecting hazardous materials or conditions, air or water pollutants, chemical agents, biological organisms or radiological conditions. The genetic and biochemical applications include testing and/or analysis of molecules, or reactions between such molecules in microfluidic devices.

In a microfluidic device, a plurality of determinations may be performed concurrently and/or consecutively. By having channels that have ultra small cross-sections, operations can be carried out with very small volumes. In addition, by having very sensitive detection systems, very low concentration of a detectable label can be employed. This allows for the use of very small samples and small amounts of reagents.

Droplet microfluidics refers to the set of technologies that are being developed for manipulating very small, substantially uniform, liquid drops, micro- to nano-liters in volume, which are supported on a solid surface, sandwiched between two solid plates or sucked into a solid channel. The manipulations include moving the droplets around, making them coalesce, and breaking them up. These technologies have a promising potential for developing commercially viable droplet-based microfluidic platforms for biotechnology and other applications. One of the reasons is that the smaller the length scale over which transport processes (convection, diffusion and reaction) take place, the faster the completion time of the process. As such, the drive toward high-throughput screening and diagnostics requires the concomitant development of associated microfluidic enabling technologies.

Droplet microfluidics may be employed in the area of biochemical and biophysical investigations of single cells. Knowledge of cell activity may also be gained by measuring and recording electrical potential changes occurring within a cell, which changes depend on the type of cells, age of the culture and external conditions such as temperature or chemical environment. Thus, precisely controlling the physical and chemical environment of a cell under study significantly enhances the value of the research. In addition, as further detailed hereinunder, cells activity can also exploited for the purpose of detecting and/or identifying chemicals in a sample.

The ability to sense, analyze, monitor and/or control transport of fluid through or from a microchannel is one of the fundamental properties required for all the above applications.

An objective in developing new techniques is not only to be able to selectively identify target compounds but to be able to assay large numbers of samples. Yet, there remain problems in detecting and measuring low levels of compounds conveniently, safely and quickly.

One of the favored approaches of analyzing, detecting and/or monitoring substances involves the use of light. Traditional methods involve illuminating a sample with light and using absorbance or scattering characteristics of the sample to analyze the analyte present therein. Typically, light based methods utilize one or more luminescent materials, such as fluorescent materials, either as labels or as reporters. Additionally, the analyte of interest can also have luminescent properties.

When a fluorescent atom or molecule absorbs light, electrons are boosted to a higher energy shell of an unstable excited state. During the lifetime of excited state (typically 1-10 nanoseconds) the fluorescent atom undergoes conformational changes and is also subject to a multitude of possible interactions with its molecular environment. The energy of excited state is partially dissipated, yielding a relaxed singlet excited state from which the excited electrons fall back to their stable ground state, emitting light of a specific wavelength. The emission spectrum is shifted towards a longer wavelength than its absorption spectrum. The difference in wavelength between the apex of the absorption and emission spectra of the fluorescent atom (also referred to as the Stokes shift), is typically small.

In a fluorescent material, not all the molecules initially excited by absorption return to the ground state by fluorescence emission. Other processes such as collisional quenching, fluorescence resonance energy transfer and intersystem crossing may also depopulate the excited state. A ratio of the number of fluorescence photons emitted to the number of photons absorbed, called “fluorescence quantum yield,” is a measure of the relative extent to which these processes occur. For fluorescent materials which are commercially available, only a small portion (about 0.1%) of the absorbed light is actually emitted.

The low fluorescence quantum yield and the small separation between the absorption and emission spectra, require the usage of spectral discrimination methods to allow a clear detection. Typically, the discrimination methods utilize a set of filters on the excitation path and emission path of a fluorescence detection system. Such filters were greatly developed during the past years, and are being manufactured by various companies.

Heretofore, attempt to developed microfluidic devices for the purpose of detecting analysts, resulted in only partial success.

U.S. Pat. No. 6,614,030 discloses an optical detection device for identifying and detecting fluorophores in during operations involving fluorescent signals. The optical detection device includes an excitation light source, and an optical setup for guiding excitation light and emission light. In use, the housing of the device is accurately moved over a small area in relation to a channel in a microfluidic device.

U.S. Pat. No. 6,602,702 discloses a system for the rapid characterization of multi-analyte fluids. The system employs a sensor array, formed of a plurality of cavities, each trapping a chemically sensitive particle, which is configured to produce light when a receptor coupled to the particle interacts with an analyte of the fluid. The analytes within the fluid are then characterized by pattern recognition techniques.

U.S. Pat. No. 6,551,838 discloses a device having a plurality of reservoirs covered by barrier layers which can be disintegrated or permeabilized to expose the isolated contents to the one or more environmental components. The reaction between the contents of the reservoirs and the environmental components generate a signal which is detected by conventional technique.

Several attempts have been made to incorporate biological materials as biosensors capable of sensing physical or chemical environmental conditions in microfluidic devices.

Generally, a biosensor is a device that qualifies and/or quantifies a physiological or biochemical signal. Biosensors have been developed to overcome some of the shortcomings of the classical analyte detection techniques. Good biosensing systems are characterized by specificity, sensitivity, reliability, portability, ability to function even in optically opaque solutions, real-time analysis and simplicity of operation. Biosensors couple a biological component with an electronic transducer and thus enable conversion of a biochemical signal into a quantifiable electrical response.

The function of the biosensor depends on the biochemical specificity of the biologically active material. Enzymes, antibodies, aptamers, DNA, receptors, organelles and microorganisms as well as plant cells or tissues have been used as biological sensing elements. The most commonly used biological element in the construction of biosensors are enzymes, due to their high specific activities as well as high analytical specificity. Purified enzymes are, however, expensive and unstable, thus limiting their applications in the field of biosensors.

The use of whole cells as the biosensing element negates the lengthy procedure of enzyme purifications, preserves the enzymes in their natural environment and protects it from inactivation by external toxicants such as heavy metals. Whole cells also provide a multipurpose catalyst especially when the process requires the participation of a number of enzymes in sequence. Whole cells have been used either in viable or non-viable form. Viable microbes, for example, can metabolize various organic compounds resulting in various end products like ammonia, carbon dioxide, acids and the like, which can be monitored using a variety of transducers [Burlage (1994) Annu. Rev. Microbiol. 48: 291-309; Riedel (1998) Anal. Lett. 31:1-12; Arikawa (1998) Mulchandani, Rogers (Eds.) Enzyme and Microbial Biosensors: Techniques and Protocols. Humanae Press, Totowa, N.J., pp. 225-235; and Simonian (1998) Mulchandani, Rogers (Eds.) Enzyme and Microbial Biosensors: Techniques and Protocols. Humanae Press, Totowa, N.J. pp: 237-248].

The selection of microbial culture which corresponds well with a spectrum of compounds present in the sample is of significant importance.

A number of selection approaches are known in the art. For example, adaptation of a microbe for induction of desirable metabolic pathways and uptake systems can be effected by cultivation in a medium containing appropriate substrates [Di Paolantonio and Rechnitz (1982) supra; Riedel (1990) Anal. Lett. 23:757-770; Fleschin (1998) Prep. Biochem. Biotechnol. 28:261-269]. Specifically, for the biochemical degradation of complex substrates such as mixtures of phenols, the use of activated sludge obtained from waste treatment plants can serve as an acclimatized mixed microbial consortium as compared to pure cultures [Joshi and D'souza (1999) J. Environ. Sci. Health Part A Eviron. Sci. Engng. 34:1689-1700].

Alternatively, when a single cell does not contain all enzymes necessary for a sequential set of reactions a mixture of microbial cultures can be used. Thus, Gluconobacter oxydans containing glucose oxidase has been used in conjunction with saccharomyces cerevisiae cells containing periplasmic invertase or permeabilized Kluyveromyces marxianus cells containing intracellular β-galactosidase, in the fabrication of a sucrose and a lactose biosensor, respectively [Svitel (1998) Biotechnol. Appl. Biochem. 27:153-[58]. Note, the major drawback of such an approach is the need to maintain at least two cultures of microorganisms on a single sensor which may prove problematic such as due to different nutritional needs.

Microbial biosensors based on light emission from luminescent bacteria are also utilized in analyte detection. Bioluminescent bacteria are found in nature, their habitat ranging from marine to terrestrial environments. Bioluminescent whole cell biosensors have also been developed using genetically engineered microorganisms for the monitoring of organic, pesticide and heavy metal contamination. The microorganisms used in these biosensors are typically produced with an exogenous plasmid into which a reporter gene under the control of an inducible promoter of interest is placed.

Following are prior art technologies incorporating biosensors in microfluidic devices.

U.S. Pat. No. 6,436,698 is directed at automatic measurement of water toxicity, using luminescent microorganisms living in freshwater. Test samples are injected using a needle into multi-well plate containing the luminescent microorganisms and, after a lapse of certain times from the injection, luminosity is detected by a sensor.

U.S. Pat. No. 6,117,643 is directed at detection of pollutants, explosives and heavy-metals. A bioreporter, capable of metabolizing a particular substance to emit light, is placed in a selectively permeable container. When the light is emitted, an optical application specific integrated circuit generates an electrical signal which indicates the concentration of the substance.

U.S. Pat. No. 6,133,046 teaches the use of a fixed electrode and a moving electrode, whereby the surfaces of the electrodes bound a ligand of the analyte to be detected (e.g., an antibody, whereby the analyte is an antigen or a hapten, a receptor whereby the analyte is a receptor, etc.). When a sample is placed between the electrodes, an electric signal is generated, depending on whether or not the analyte is present.

Additional prior art of relevance include: U.S. Pat. Nos. 6,638,752, 6,638,483, 6,636,752, 6,632,619, 6,627,433, 6,630,353, 6,620,625, 6,544,729, 6,537,498, 6,521,188, 6,453,928, 6,448,064, 6,340,572 and 5,922,537.

The above technologies suffer from many limitations. For example, in most prior art systems, the optical setup which is large, bulky and generally unsuitable for field use. In addition, there is the problem of obtaining a reliable optical signal, in effect compromising maximizing the signal from the detectable material while minimizing the background signal. Furthermore, in prior art systems which are based on mechanical scan (e.g., moving electrode, moving light ray or moving sample), inaccurate readings may occur due to misalignment of the various components. With respect to the sensing process, it is difficult to generate transport of the sample in the channels and to distinguish between signals arriving from different locations.

There is thus a widely recognized need for, and it would be highly advantageous to have a method and system for identifying and/or detecting chemical agents and biological materials, devoid of the above limitations. The present invention provides solutions to the problems associated with prior art techniques aimed at multiplexed detection of a plurality of analytes.

### SUMMARY OF THE INVENTION

According to one aspect of the present invention there is provided a device for detecting a presence of an analyte in a sample. The device comprising a device body configured with at least one reaction chamber configured for containing a sensor capable of producing a detectable signal when exposed to the analyte in the sample, the at least one reaction chamber being in fluid communication with at least one sample port and at least one actuator port via a first set of microfluidic channels arranged such that application of a negative pressure to the at least one actuator port delivers fluid placed in the at least one sample port to the at least one reaction chamber.

According to further features in preferred embodiments of the invention described below, the at least one reaction chamber is configured so as to enable sustaining a negative pressure environment within the at least one reaction chamber.

According to still further features in the described preferred embodiments the at least one reaction chamber is configured such that the material placed therein does not substantially obstruct fluid flow in and out of the at least one reaction chamber.

According to still further features in the described preferred embodiments the microfluidic channels of the first set of microfluidic channels are connected to the at least one reaction chamber substantially above a bottom surface thereof.

According to still further features in the described preferred embodiments the at least one reaction chamber includes a plurality of reaction chambers sequentially interconnected via a second set of fluidic microchannels.

According to still further features in the described preferred embodiments the device further comprises a pumping device for generating the negative pressure.

According to still further features in the described preferred embodiments the device further comprises a sample reservoir being in fluid communication with the sample port.

According to still further features in the described preferred embodiments the device body is capable of allowing transmission of light having a predetermined wavelength therethrough.

According to still further features in the described preferred embodiments the device body comprises a material selected from the group consisting of silicon, plastic and glass.

According to still further features in the described preferred embodiments the device further comprises at least one humidity sensor, adapted for being positioned in the at least one reaction chamber, the humidity sensor being capable of generating a detectable signal when a level of humidity in the at least one reaction chamber is above a predetermined threshold.

According to still further features in the described preferred embodiments at least a portion of the plurality of chambers comprises different sensors, each capable of generating a detectable signal when exposed to a different analyte of the at least one analyte.

According to another aspect of the present invention there is provided a device for detecting at least one analyte present in a sample, the device comprising at least one array of reaction chambers, each array having a plurality of reaction chambers, sequentially interconnected by a plurality of fluid channels, in a manner such that each reaction chamber is in direct fluid communication with at least two other reaction chambers, whereby a first reaction chamber of the at least two other reaction chambers serves as a fluid source and a second reaction chamber of the at least two other reaction chambers serves as a fluid sink, wherein each reaction chamber is designed for containing a sensor capable of generating a detectable signal when exposed to the at least one analyte.

According to further features in preferred embodiments of the invention described below, at least a portion of the sensors are biological sensors.

According to yet another aspect of the present invention there is provided a device for detecting at least one analyte present in a sample, the device comprising a substrate configured with: (a) a plurality of chambers for holding a fluorescent sensor and incubating reaction between the fluorescent sensor and the at least one analyte; (b) a plurality of fluid channels interconnecting at least a portion of the plurality of chambers; and (c) a plurality of waveguides designed and constructed to distribute excitation light among the plurality of chambers in a manner such that impingement of the excitation light on the fluorescent sensor is maximized and impingement of the excitation light on a surface of the substrate is minimized.

According to further features in preferred embodiments of the invention described below, the substrate is made of a disposable material.

According to still further features in the described preferred embodiments the plurality of waveguides are integrated with or formed in the substrate.

According to still further features in the described preferred embodiments the substrate is formed with a plurality of grooves, sizewise compatible with the plurality of waveguides, and further wherein at least a portion the plurality waveguides are designed insertable to and/or detachable of at least a portion of the plurality of grooves.

According to still further features in the described preferred embodiments the plurality of waveguides are arranged in a multi-furcated arrangement.

According to still further features in the described preferred embodiments the multi-furcated arrangement comprises a plurality of light splitting junctions, each capable of redirecting the excitation light into at least one of the plurality of waveguides.

According to still further features in the described preferred embodiments the plurality of waveguides are capable of imposing at least one predetermined propagation direction on the excitation light.

According to still further features in the described preferred embodiments the device further comprises at least one additional optical element, capable of imposing at least one predetermined propagation direction on the excitation light.

According to still further features in the described preferred embodiments the at least one predetermined propagation direction is substantially parallel to the surface of the substrate.

According to still further features in the described preferred embodiments the at least one additional optical element is selected from the group consisting of a diffraction grating, a reflection grating and a mini-prism.

According to still further features in the described preferred embodiments at least one of the plurality of chambers comprises a reflective coat, covering at least one internal wall of the chamber.

According to still further features in the described preferred embodiments the reflective coat is wavelength selective.

According to still further features in the described preferred embodiments the device further comprises a selective filter positioned on or close to the substrate and capable of prevention transmission of the excitation light therethrough.

According to still further features in the described preferred embodiments the device further comprises a plurality of optical focusing devices positioned so as to focus or collimate optical signals generated by the florescent sensor in response to the excitation light.

According to still further features in the described preferred embodiments the plurality of optical focusing devices are selected from the group consisting of microlenses and diffraction gratings.

According to still further features in the described preferred embodiments the fluid channels are microfluidic channels.

According to still further features in the described preferred embodiments the device further comprises a pump interface connectable to a pumping device.

According to still further features in the described preferred embodiments the device further comprises the pumping device.

According to still further features in the described preferred embodiments the device further comprises an electronic circuitry designed and constructed for controlling the plurality of micro-pumps.

According to still further features in the described preferred embodiments the plurality of fluid channels are connected to the plurality of reaction chamber substantially above a bottom surface thereof.

According to still another aspect of the present invention there is provided a device for detecting at least one analyte present in a sample, the device comprising a plurality of reaction chambers and a plurality of channels, interconnecting at least a portion of the plurality of reaction chambers, wherein each one of the plurality of reaction chambers comprises a biological sensor, capable of generating a detectable signal when exposed to the at least one analyte.

According to further features in preferred embodiments of the invention described below, the plurality of chambers are addressable, hence allow imaging thereof.

According to still further features in the described preferred embodiments at least a portion of the plurality of chambers comprises different biological sensors, each capable of generating a detectable signal when exposed to a different analyte of the at least one analyte.

According to still further features in the described preferred embodiments the plurality of reaction chambers are configured such that the biological sensor does not substantially obstruct fluid flow in and out of the plurality of reaction chambers.

According to still further features in the described preferred embodiments a body of the device is capable of allowing transmission of light having a predetermined wavelength therethrough.

According to an additional aspect of the present invention there is provided a system for detecting at least one analyte present in a sample, the system comprising: a detecting device having a plurality of reaction chambers and a plurality of channels interconnecting at least a portion of the plurality of reaction chambers, wherein at least a portion of the plurality of reaction chambers comprises a sensor, capable of generating a detectable optical signal when exposed to the at least one analyte; a planar light detector capable of receiving optical signals from the detecting device and providing an image of sensors generating the optical signals.

According to further features in preferred embodiments of the invention described below, the system further comprises a data processor, supplemented by an algorithm for receiving image information from the planar light detector and determining presence of the at least one analyte.

According to still further features in the described preferred embodiments the system further comprises a control unit for sending control signals to the detecting device.

According to still further features in the described preferred embodiments the system further comprises a temperature control unit for controlling a temperature of the detecting device and/or the planar light detector.

According to still further features in the described preferred embodiments the algorithm is capable of determining concentration of the at least one analyte.

According to still further features in the described preferred embodiments the plurality of reaction chambers are configured so as to enable sustaining a negative pressure environment within the plurality of reaction chambers.

According to still further features in the described preferred embodiments the plurality of reaction chambers are configured such that the sensor does not substantially obstruct fluid flow in and out of the plurality of reaction chambers.

According to still further features in the described preferred embodiments the plurality of channels are connected to the plurality of reaction chamber substantially above a bottom surface thereof.

According to still further features in the described preferred embodiments at least a portion of the plurality of reaction chambers are sequentially interconnected via at least a portion of the channels.

According to still further features in the described preferred embodiments a body of the detecting device is capable of allowing transmission of light having a predetermined wavelength therethrough.

According to still further features in the described preferred embodiments the body comprises a material selected from the group consisting of silicon, plastic and glass.

According to still further features in the described preferred embodiments the sensor is a biological sensor.

According to still further features in the described preferred embodiments the planar light detector comprises a matrix having a plurality of addressable elementary units, each being capable of converting the optical signal into an electrical signal.

According to still further features in the described preferred embodiments the elementary units of the planar light detector are selected from the group consisting of positive-intrinsic-negative photodiodes, avalanche photodiodes, silicon chips and photomultipliers.

According to still further features in the described preferred embodiments the planar light detector is selected from the group consisting of a CCD camera and a CMOS detector.

According to still further features in the described preferred embodiments the system further comprises a light source for emitting excitation light so as to excite the sensor to thereby emit the optical signal.

According to still further features in the described preferred embodiments the light source comprises a light emitting diode.

According to still further features in the described preferred embodiments the light emitting diode is coupled to a collimator capable of redirecting the excitation light to form a substantially collimated light beam.

According to still further features in the described preferred embodiments the light source comprises an arrangement of light emitting diodes.

According to still further features in the described preferred embodiments each light emitting diode of the arrangement of light emitting diodes is coupled to a collimator capable of redirecting the excitation light to form a substantially collimated light beam.

According to still further features in the described preferred embodiments the system further comprises a temperature control unit for controlling a temperature of the detecting device, the planar light detector and/or the light source.

According to still further features in the described preferred embodiments the temperature control unit is selected from the group consisting of a thermoelectric device, a liquid cooler, a gas cooler and a blower.

According to still further features in the described preferred embodiments the system further comprises at least one selective filter positioned between the detecting device and the planar light detector, the at least one selective filter being capable of transmitting the optical signals and preventing transmission of the excitation light.

According to still further features in the described preferred embodiments the system further comprises a plurality of optical fibers for guiding the excitation light into the detecting device.

According to still further features in the described preferred embodiments the system further comprises an optical focusing device for focusing the optical signal on the planar light detector.

According to still further features in the described preferred embodiments the optical focusing device is a video lens.

According to still further features in the described preferred embodiments the system further comprises at least one opaque object, positioned between the detecting device and the planar light detector, wherein the optical focusing device is configured to focus the excitation light on the at least one opaque object thereby to substantially prevent impingement of the excitation light on the planar light detector.

According to still further features in the described preferred embodiments the system further comprises at least one reflector, positioned between the detecting device and the planar light detector, wherein the optical focusing device is configured to focus the excitation light on the at least one reflector so that the excitation light is reflected back to at least one of the plurality of reaction chambers.

According to still further features in the described preferred embodiments the system further comprises a transport mechanism for actuating transport of a sample fluid in the plurality of fluid channels, thereby to fill the plurality of reaction chambers with the sample fluid.

According to still further features in the described preferred embodiments the system further comprises a draining system and further wherein the transport mechanism is capable of maintaining a continues flow of the sample fluid in the plurality of fluid channels thereby to continuously replace the sample fluid in the plurality of reaction chambers.

According to still further features in the described preferred embodiments the planar light detector is capable of providing the image substantially in real time.

According to still further features in the described preferred embodiments a portion of the plurality of reaction chambers comprises a material capable of generating a detectable reference optical signal at all times.

According to still further features in the described preferred embodiments the transport mechanism comprises a pumping device, capable of generating a negative pressure in the plurality of reaction chambers and the plurality of fluid channels.

According to still further features in the described preferred embodiments the pumping device comprises a plurality of micro-pumps.

According to still further features in the described preferred embodiments the system further comprises an electronic circuitry designed and constructed for controlling the plurality of micro-pumps.

According to still further features in the described preferred embodiments the electronic circuitry comprises at least one feedback line for monitoring operation and/or status of the plurality of micro-pumps.

According to still further features in the described preferred embodiments the transport mechanism further comprises a vacuum chamber connected to the pumping device and capable of maintaining a negative pressure environment.

According to still further features in the described preferred embodiments the transport mechanism further comprises a pressure sensor for sensing a pressure at an inlet of the vacuum chamber.

According to still further features in the described preferred embodiments the transport mechanism further comprises a flow sensor for sensing flow parameters of the sample fluid.

According to still further features in the described preferred embodiments the transport mechanism further comprises at least one tap for controlling the flow parameters.

According to still further features in the described preferred embodiments the transport mechanism further comprises at least one valve for activating and deactivating the transport of the sample fluid.

According to still further features in the described preferred embodiments the transport mechanism further comprises a hydrophobic filter for protecting at least one component of the transport mechanism.

According to still further features in the described preferred embodiments the system further comprises electronic circuitry for controlling flow rate of the sample fluid.

According to still further features in the described preferred embodiments the electronic circuitry is designed and constructed to allow equal filling of the sample fluid in the plurality of reaction chambers.

According to still further features in the described preferred embodiments the transport mechanism comprises an electric field generator, for generating a non-uniform electric field capable of inducing polarization on molecules of the sample fluid, hence to fill the plurality of reaction chambers with the sample fluid via dielectrophoresis.

According to still further features in the described preferred embodiments the transport mechanism comprises a column of the sample fluid, the column having a height selected such that a hydrostatic pressure, generated at a bottom of the column, is sufficient for actuating the transport of the sample fluid.

According to still further features in the described preferred embodiments the plurality of fluid channels are designed and constructed such that fluid sample flows therethrough via capillary forces.

According to still further features in the described preferred embodiments the sample is a liquid sample.

According to still further features in the described preferred embodiments the sample is a gas sample.

According to still further features in the described preferred embodiments the system further comprises a mechanism for binding components of the gas sample to an aqueous phase.

According to yet an additional aspect of the present invention there is provided an apparatus for imaging a pattern of optical signals received from a fluorescent material arranged in a plurality of predetermined locations, the apparatus comprising: (a) a planar light detector engaging a first plane; (b) a optical element engaging a second plane substantially parallel to the first plane; (c) a light source interposed between the first and the second planes, the light source capable of generating excitation light in a direction other than a direction of the planar light detector; the optical element and the planar light detector being designed an constructed such that the excitation light is collimated by the optical element and impinges on at least a portion of the plurality of predetermined locations, and emission light, emitted by the fluorescent material in response to the excitation light, is focused by the optical element and impinges on the planar light detector, to form the pattern of the optical signal thereupon.

According to further features in preferred embodiments of the invention described below, the optical element comprises a plurality of lenses.

According to still further features in the described preferred embodiments the light source comprises a plurality of light emitting devices.

According to still further features in the described preferred embodiments an arrangement of the plurality of lenses is compatible with an arrangement of the plurality of predetermined locations.

According to still further features in the described preferred embodiments an arrangement of the plurality of light emitting devices is compatible with the arrangement of the plurality of lenses.

According to still further features in the described preferred embodiments the apparatus further comprises an infrared filter positioned between the planar light detector and the light source.

According to still further features in the described preferred embodiments the apparatus further comprises an additional optical element positioned between the light source and the planar light detector, the additional optical being capable of preventing the excitation light from impinging on the planar light detector.

According to still further features in the described preferred embodiments the additional optical element comprises at least one opaque object.

According to still further features in the described preferred embodiments the additional optical element comprises at least one reflector.

According to still further features in the described preferred embodiments a shape of the reflector is selected so as to direct the excitation light in a direction of the optical element engaging the first plane.

According to still further features in the described preferred embodiments each of the plurality of light emitting devices is positioned at a focal point of one lens of the plurality of lenses.

According to still an additional aspect of the present invention there is provided a method of determining concentration of an analyte from optical signals recorded of a reaction chamber in response to excitation light, the reaction chamber containing a plurality of biological sensors producing a fluorescent material when exposed to the analyte, the method comprising: (a) defining a plurality of slices, each slice having at least one biological reporter; (b) for each slice, representing the at least one biological reporter as at least one equivalent light emitter, located at a predetermined location within the slice, and calculating local radiation contribution emitted by the at least one equivalent light emitter; and (c) integrating the local radiation contribution over the plurality of slices so as to obtain an integrated radiation intensity; and (d) using the recorded optical signals and the integrated radiation intensity for determining the concentration of the analyte.

According to further features in preferred embodiments of the invention described below, the calculation of the local radiation contribution comprises calculating effective quantum efficiency and at least one transmission coefficient corresponding to the excitation light and light emitted by the at least one equivalent light emitter.

According to still further features in the described preferred embodiments the effective quantum efficiency comprises emission effective quantum efficiency and excitation effective quantum efficiency.

According to still further features in the described preferred embodiments the determination of the concentration of the analyte is by calculating an occupation area of the fluorescent material, the occupation area being defined as a projection of an occupation volume on a plane perpendicular to a direction of the excitation light.

According to a further aspect of the present invention there is provided a method of detecting analytes in a sample fluid, comprising: (a) providing a device having a plurality of reaction chambers and a plurality of channels, interconnecting at least a portion of the plurality of reaction chambers, wherein each one of the plurality of reaction chambers comprises a biological sensor, capable of generating a detectable signal when exposed to the at least one analyte; (b) filling at least a first portion of the plurality of reaction chambers with the sample fluid; (c) generating a condition for the biological sensor to generate the detectable signal; and (d) detecting the detectable signal thereby detecting the analytes in the sample fluid.

According to further features in preferred embodiments of the invention described below, step (b) is by generating a negative pressure.

According to still further features in the described preferred embodiments step (b) is by dielectrophoresis.

According to still further features in the described preferred embodiments step (b) is by capillary transport.

According to still further features in the described preferred embodiments step (b) is by injection.

According to still further features in the described preferred embodiments the detectable signal is selected from the group consisting of a detectable optical signal, a detectable electrical signal and a detectable electrochemical signal.

According to still further features in the described preferred embodiments the biological sensors is capable of producing a bioluminescent material.

According to still further features in the described preferred embodiments the biological sensors is capable of producing a phosphorescent material.

According to still further features in the described preferred embodiments the biological sensor is capable producing a fluorescent material.

According to still further features in the described preferred embodiments step (c) comprises irradiating the biological sensor by excitation light.

According to still further features in the described preferred embodiments the method further comprises filling different portions of the plurality of reaction chambers with different fluids.

According to still further features in the described preferred embodiments at least one of the different fluids has a known composition, hence serving as a control fluid.

According to still further features in the described preferred embodiments different portions of the plurality of reaction chambers comprise different biological sensors.

According to still further features in the described preferred embodiments the method further comprises exposing each one of the biological sensors to at least two of the different fluids.

According to still further features in the described preferred embodiments the method further comprises generating an image of the reaction chambers.

According to still further features in the described preferred embodiments the method further comprises controlling a temperature of the detecting device.

According to still further features in the described preferred embodiments the controlling the temperature is by a thermoelectric device.

According to still further features in the described preferred embodiments the controlling the temperature is by a liquid cooler.

According to still further features in the described preferred embodiments the controlling the temperature is by a blower.

According to still further features in the described preferred embodiments the method further comprises using the detectable signal for determining a concentration of the analyte.

According to still further features in the described preferred embodiments the detecting device is disposable.

According to still further features in the described preferred embodiments step (d) is by a matrix having a plurality of addressable elementary units, each being capable of converting the optical signal into an electrical signal.

According to still further features in the described preferred embodiments the irradiation is by a light emitting diode.

According to still further features in the described preferred embodiments the method further comprises redirecting the excitation light to form a substantially collimated light beam.

According to still further features in the described preferred embodiments the irradiation is by an arrangement of light emitting diodes.

According to still further features in the described preferred embodiments the method further comprises a plurality of optical fibers for guiding the excitation light into the detecting device.

According to still further features in the described preferred embodiments the method further comprises focusing the optical signal prior to step (d).

According to still further features in the described preferred embodiments the optical focusing device comprises a plurality of lenses positioned to substantially prevent cross talks between different optical signals of different sensors.

According to still further features in the described preferred embodiments the method further comprises maintaining continues flow of the sample fluid in the plurality of fluid channels thereby to continuously replace the sample fluid in the plurality of reaction chambers.

According to still further features in the described preferred embodiments the method further comprises providing an image of the plurality of reaction chambers substantially in real time.

According to still further features in the described preferred embodiments a portion of the plurality of reaction chambers comprises a biological material capable of generating a detectable reference optical signal at all times.

According to still further features in the described preferred embodiments the method further comprises binding components of the gas to an aqueous phase.

According to yet a further aspect of the present invention there is provided a method of dehydrating a biological material, the method comprising: providing a first set of chambers for holding the biological material, and a second set of chambers having at least one fluid channel formed therein; placing the first set of chambers and the second set of chambers in a negative pressure environment so as to dehydrate the biological material; and positioning the second set of chambers on the first set of chambers so as seal the first set of chambers hence to maintain the negative pressure in the first and the second sets of chambers.

According to further features in preferred embodiments of the invention described below, each of the second set of chambers comprises a window for allowing evaporation of liquids therethrough.

According to still further features in the described preferred embodiments the method further comprises pressing the second set of chambers on the first set of chambers, such that when a respective chamber of the second set of chambers is pressed on a respective chamber of the first set of chambers a respective window is sealed.

According to still further features in the described preferred embodiments the pressing is done so as not to obstruct the at least one fluid channel.

According to still further features in the described preferred embodiments the method further comprises immobilizing the biological material to each of the first set of chambers, prior to the step of placing the first and the second sets of chambers in the negative pressure environment.

According to still further features in the described preferred embodiments the immobilizing is by encapsulating the biological sensor into a meltable membrane.

According to still further features in the described preferred embodiments the immobilizing is by encapsulating the biological material into a meltable membrane.

According to still further features in the described preferred embodiments the immobilizing is by a material selected from the group consisting of agar, alginate, poly-vinyl alcohol, sol-gel and carraginan.

According to still further features in the described preferred embodiments the biological sensors comprises a population of cells, the population of cells including a reporter expression construct being expressible in a cell of the population when exposed to the analyte.

According to still further features in the described preferred embodiments the population of cells is eukaryotic cells.

According to still further features in the described preferred embodiments the population of cells is prokaryotic cells.

According to still further features in the described preferred embodiments each of the reporter expression construct includes a cis-acting regulatory element being operably fused to a reporter gene.

According to still further features in the described preferred embodiments the reporter gene is selected from a group consisting of a fluorescent protein, an enzyme and an affinity tag.

According to still further features in the described preferred embodiments the cis-acting regulatory element is a promoter.

According to still further features in the described preferred embodiments the promoter is selected from the group consisting of MipA, LacZ, GrpE, Fiu, MalPQ, oraA, nhoA, recA, otsAB and yciD.

According to still further features in the described preferred embodiments the cis-acting regulatory element is stress regulated.

According to still further features in the described preferred embodiments the analyte is selected from the group consisting of a condition and a substance.

According to still further features in the described preferred embodiments the condition is selected from the group consisting of a temperature condition and a radiation condition.

According to still further features in the described preferred embodiments the substance is a naturally occurring product or a synthetic product.

According to still further features in the described preferred embodiments the populations of cells is tagged.

The present invention successfully addresses the shortcomings of the presently known configurations by providing a method, device and system for detecting and/or identifying analytes.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are schematic illustrations of a bottom view (FIG. 1a) and a top view (FIG. 1b) of a device for detecting one or more analytes, according to a preferred embodiment of the present invention;

FIG. 1c is a schematic illustration of a side view of one reaction chamber of the device of FIGS. 1a-b, according to a preferred embodiment of the present invention;

FIG. 2a-b are schematic illustrations of the reaction chambers and the fluid channels of the device in a preferred embodiment in which the chambers and the channels are arranged in one or more sequential arrays;

FIG. 3a is a schematic illustration of the device in the preferred embodiment in which a plurality of micro-pumps is employed;

FIG. 3b is a schematic illustration of one micro-pump, according to a preferred embodiment of the present invention;

FIG. 3c is a schematic diagram exemplifying a configuration of an electronic circuitry controlling the micro-pump of the device shown in FIG. 3a, according to a preferred embodiment of the present invention;

FIG. 4 is a transport mechanism in a preferred embodiment in which external pumping is utilized, according to a preferred embodiment of the present invention;

FIG. 5 is a simplified illustration of a portion of the device in which the transport mechanism comprises an electric field generator, according to a preferred embodiment of the present invention;

FIG. 6 is a simplified illustration of a portion of the device in a preferred embodiment in which the transport of the sample fluid is generated by hydrostatic pressure;

FIG. 7 is a schematic illustration of a fluid channel in a preferred embodiment in which the sample fluid flows via capillary forces;

FIGS. 8a-c, are simplified illustrations of a system for detecting the analyte using optical signal, according to a preferred embodiment of the present invention;

FIG. 9 is a simplified illustration of a light detector, according to a preferred embodiment of the present invention;

FIGS. 10a-d are simplified illustrations of the device in a preferred embodiment in which the spatial separation of the excitation light from the optical signal is employed, using a plurality of waveguides;

FIG. 11a is a simplified illustration of a side view of a light source, in a preferred embodiment in which waveguides are employed;

FIG. 11b is a simplified illustration of a top view of a top view the light beam outputted by the light source, according to a preferred embodiment of the present invention;

FIG. 12 is a simplified illustration of system in a preferred embodiment in which the device is positioned between the light source and the light detector;

FIG. 13 is a schematic illustration of the system in a preferred embodiment in which a plurality of external optical fibers is employed;

FIG. 14 is a simplified illustration of the light path of the excitation light once entering the reaction chamber, according to a preferred embodiment of the present invention (FIG. 14 is rotated anticlockwise by 90° relative to FIG. 13);

FIG. 15 is a schematic illustration of a video-lens, which can be used as a focusing device, according to a preferred embodiment of the present invention;

FIGS. 16a-c are schematic illustrations of a light source of the system of FIG. 13, according to a preferred embodiment of the present invention;

FIG. 17a is a simplified illustration of the light source in a preferred embodiment in which the light source is an arrangement of light emitting devices;

FIGS. 17b-c are simplified illustrations of the system in a preferred embodiment in which the light source of FIG. 17a is positioned between the device and the light detector;

FIG. 18 is a flowchart diagram of a method of detecting analytes in a sample fluid, according to a preferred embodiment of the present invention;

FIG. 19 is a schematic illustration of a logical and physical division of the device, according to a preferred embodiment of the present invention;

FIGS. 20a-c are schematic illustration of method steps for dehydrating a biological material, according to a preferred embodiment of the present invention;

FIG. 21 is a flowchart diagram of a method of determining the concentration of the analyte from the optical signals, according to a preferred embodiment of the present invention;

FIGS. 22a-b are electronic diagrams of a CMOS detector, which can be used as a light detector, according to a preferred embodiment of the present invention;

FIG. 23a is a schematic illustration of the slicing technique of the method of FIG. 21, according to a preferred embodiment of the present invention;

FIG. 23b is a schematic illustration of an equivalent light emitter which can be used in the slicing technique of the method of FIG. 21, according to a preferred embodiment of the present invention;

FIG. 23c is a schematic illustration of the spreading of an optical signal through an aperture of the reaction chamber, according to a preferred embodiment of the present invention;

FIG. 24 is a schematic calculation diagram which can be implemented for the calculation of optical coefficients, according to a preferred embodiment of the present invention;

FIG. 25 illustrates light propagation from the equivalent light emitter to a lens, according to a preferred embodiment of the present invention;

FIG. 26 illustrates the scattering solid angle of the emitted light rays, according to a preferred embodiment of the present invention; and

FIGS. 27a-d show results of an experiment performed using the device of FIG. 3a with fresh biological sensors.

### DESCRIPTION OF THE PREFERRED EMBODIMENTS

The present invention is of a method, device and system for detecting and/or identifying analytes, which can be used for measuring fluid toxicity. Specifically, the present invention can be used to measure and analyze water or air toxicity using biological sensors, such as, but not limited to, bacteria. The present invention is further of an apparatus for reading data of a chip incorporating the biological sensors, and a method of determining the concentration the analytes based on the obtained data.

The principles and operation of a device, apparatus and system for detecting and/or identifying analytes according to the present invention may be better understood with reference to the drawings and accompanying descriptions.

Before explaining at least one embodiment of the invention in detail, it is to be understood that the invention is not limited in its application to the details of construction and the arrangement of the components set forth in the following description, illustrated in the drawings or exemplified by the Examples. The invention is capable of other embodiments or of being practiced or carried out in various ways. Also, it is to be understood that the phraseology and terminology employed herein is for the purpose of description and should not be regarded as limiting.

According to one aspect of the present invention, there is provided a device for detecting one or more analytes, generally referred to herein as device 10.

As used herein the term “analyte” refers to a molecule or a mixture of molecules in a liquid, gaseous or aerosol medium. It will be appreciated that molecules can be completely soluble in a liquid medium, alternatively they may be in a colloidal state. Thus analytes in liquid medium may be in solution or carried by the liquid medium.

Examples of analytes include, but are not limited to, small molecules such as naturally occurring compounds (e.g., compounds derived from plant extracts, microbial broths, and the like) or synthetic compounds having molecular weights of less than about 10,000 daltons, preferably less than about 5,000 daltons, and most preferably less than about 1,500 daltons, electrolytes, metals, peptides, nucleotides, is saccharides, fatty acids, steroids and the like.

As used herein the term “about” refers to ±10%.

Analytes typically include at least one functional group necessary for biological interactions (e.g., amine group, carbonyl group, hydroxyl group, carboxyl group).

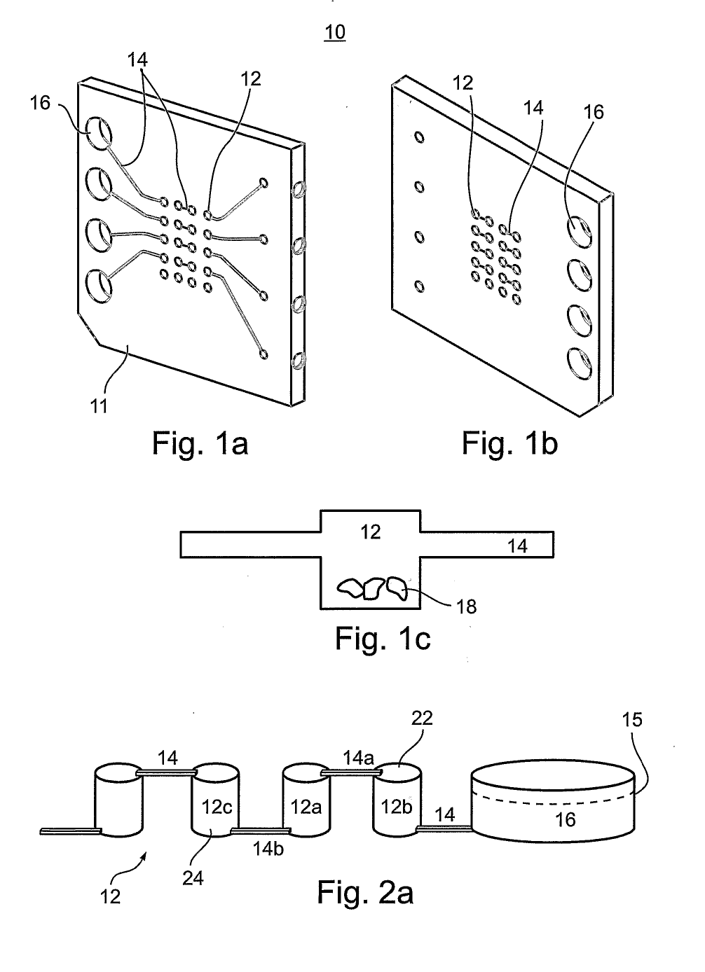

Referring now to the drawings, FIGS. 1a-b illustrate a bottom view (FIG. 1a) and a top view (FIG. 1b) of device 10, according to a preferred embodiment of the present invention. In its simplest configuration, device 10 comprises a body or substrate 11, a plurality of reaction chambers 12 and a plurality of channels 14, interconnecting at least a portion of reaction chambers 12. Reaction chambers 12 and channels 14 can be formed in or integrated with substrate 11 in any fabrication method known in the art. Device 10 is preferably disposable. Thus, substrate 11 preferably comprises a disposable material, such as, but not limited to, silicon, plastic and glass.

FIG. 1c illustrates a side view of one of reaction chambers 12. Reaction chamber 12 preferably comprises a sensor 18, capable of generating a detectable signal when exposed to the at least one analyte. According to a preferred embodiment of the present invention any detectable signal can be generated by sensors 18, depending on the type of sensor being utilized. Representative examples of detectable signals include, without limitation, optical, electrical and electrochemical signals. When the detectable signals are optical, substrate 11 is preferably made transparent to the optical signals. Substrate 11 can also be manufactured so as to selectively allow transmission of light having a predetermined wavelength. This can be done, for example, by doping substrate 11 by an impurity, whereby the type and concentration of the impurity is selected in accordance with the wavelength of the optical signals.

Device 10 further comprises one or more sample ports 16, being in fluid communication with chambers 12 via channels 14. Sample ports 16 serve for feeding sample fluid 15 (gas or liquid) into channels 14 through which sample fluid 15 flows into reaction chambers 12 and interacts with the sensors. When sample fluid 15 is in gaseous state, its components (e.g., organic components) are preferably bound to an aqueous phase prior to the feeding of sample port 16.

Given that a sufficient number of sensors are utilized, the intensity of the signal, generated by population of sensors 18, typically depends on the amount of detectable analyte being in contact therewith. For a given sample fluid having a given concentration, the intensity of the signal is proportional to the amount of sample fluid present in reaction chambers 12. To optimize correlation between the intensity of the signal and the concentration of the analyte in sample fluid 15, reaction chambers 12 are preferably of substantially equal volume. The advantage of this embodiment is that when chambers 12 are filled, the occupation ratio between the analyte and sensors 18 does not vary from one chamber to another, so that, once a functional relationship between the intensity of the signal and the concentration of analyte is established (e.g., using a simple calibration curve, a mathematical model, etc.), the same functional relationship can be used for many reaction chambers.

Reaction chambers 12 are preferably addressable so as to allow imaging thereof, as further detailed hereinunder. In this embodiment, at least a few of reaction chambers 12 may comprise different sensors, each capable of generating the detectable signal when exposed to a different analyte.

Device 10 is typically of small dimensions. Preferably, the area of device 10 is less than about 10 cm², more preferably less than 1 cm², most preferably less than 0.1 cm². The number of reaction chambers and fluid channels of device 10 is not limited, and may vary from a few to a few hundreds of thousands of reaction chambers positioned on the same device. Thus, device 10 is preferably a microfluidic device so as to facilitate forming therein or integrating therewith a large number of reaction chambers and fluid channels. When reaction chambers 12 comprise different sensors, device 10 is capable of providing a multiplexed detection of an enormous amount of different analytes.

Fluid channels 14 are preferably microfluidic channels. Transport of sample 15 from sample port 16 through channels 14 and into reaction chambers 12 can be effected using a variety of methods which are known in the art. Preferably sample transport is effected in a manner which enables provision of an equal fluid volume to each of reaction chambers 12. This may be done by a judicious positioning of fluid channels 14 and reaction chambers 12, as further detailed hereinunder.

Before providing a further detailed description of device 10, as delineated hereinabove and in accordance with the present embodiment, attention will be given to the potential applications offered thereby.

Hence, device 10 can be employed in a variety of applications. For example, in the environmental field, device 10 can be employed to detect the presence of pollutants such as halogenated hydrocarbons (used as pesticides), polycyclic aromatic hydrocarbons (carcinogenic compounds), acrylamide, acrylic acid and acrylonitrile, organophosphorous compounds (used as pesticides, insecticides, and chemical warfare agents), nitroaromatic compounds, such as nitrophenols, picric acid, trinitrotoluene (used as xenobiotics present in wastes of chemical armament plants as in civil factories for dye, pesticide, and other chemical manufacturing).

Alternatively, device 10 can be employed in the food and fermentation industries, where there is a need for quick and specific analytical tools. Analysis is needed for monitoring nutritional parameters, food additives, food contaminants, microbial counts, shelf life assessment, compliance with specifications or regulations, and other olfactory properties like smell and odor.

In pharmaceuticals and medicine, device 10 can be used for drug identification and qualification (e.g., determination of active ingredients in pharmaceutical formulations]. Device 10 can also be used for detecting narcotics and explosives such as trinitrotoluene (TNT), cyclonite (RDX), pentaerythritol tetranitrate (PETN) C-4 class explosives, and combinations thereof [Yinon, Y. and Zitrin, S. (1993) Modern Methods and Applications in Analysis of Explosives, John Wiley & Sons, Ltd., Sussex, U. K.].

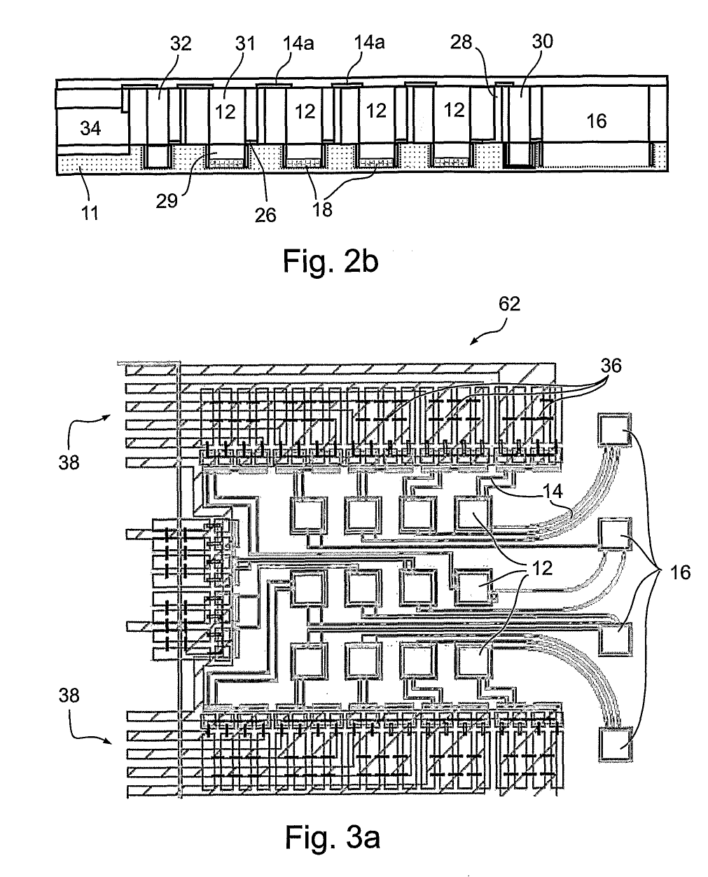

Referring now again to the drawings, FIGS. 2a-b are schematic illustrations of reaction chambers 12 and fluid channels 14, according to a preferred embodiment of the present invention. In an embodiment in which chambers 12 and channels 14 are arranged in one or more sequential arrays.

It is to be understood that the configurations shown in FIGS. 2a-b are not to be considered as limiting and that other arrangements of chambers 12 and channels 14, such as, but not limited to, arrangement facilitating equal filling, are not excluded from the scope of the present invention.

Hence, each reaction chamber in an array (except the first and the last) is preferably in direct fluid communication with at least two other reaction chambers. For example, referring to FIG. 2a, chamber 12a is in direct fluid communication with chambers 12b and 12c, such that chamber 12a is fed from chamber 12b and drained through chamber 12c. In other words, chamber 12b serves as a fluid source for chamber 12a and chamber 12c serves as a fluid sink for chamber 12a. One of ordinary skill in the art would appreciate that with such configuration, and an appropriate transport mechanism, all the reaction chambers in the array are equally filled. For an array of equal-volume reaction chambers, this embodiment ensures equal reaction conditions in all the chambers in the array. In the configuration shown in FIG. 2a, chamber 12a is connected via channel 14a to a top surface 22 of chamber 12b and via channel 14b to a bottom surface 24 of chamber 12c. However, this need not necessarily be the case, since, for some applications, it may be desired to feed and drain all chambers at the same height-level.

FIG. 2b is a schematic illustration of an array of reaction chambers in a preferred embodiment in which chambers 12 are drained through channel 14a (connected at top surface 22) and fed through a channel 14c connected at position 26 which is located above a bottom surface 24 of chambers 12, between a lower part 29 and an upper part 31 thereof. Channels 14a and 14c are in fluid communication preferably via an additional fluid channel, designated 28 in FIG. 2b. Channel 28 can be vertical or can have any orientation with respect to channels 14a and 14c provided the fluid communication therebetween is preserved. As shown in FIG. 2b, in the presently preferred embodiment of the invention, sensors 18 are positioned at bottom surface 24, below positioned 26 so that fluid flow is not substantially obstructed. Device 10 may further comprise an input buffer 30, an output buffer 32 and/or an actuator port 34. Input buffer 30 is preferably in fluid communication with sample port 16 and serves as a fluid source for the first reaction chamber in the array. Output buffer 32 is preferably in fluid communication with actuator port 34 and serves as fluid sink for the last reaction chamber in the array. Actuator port 34 can be used to facilitate fluid transport as further detailed hereinbelow.

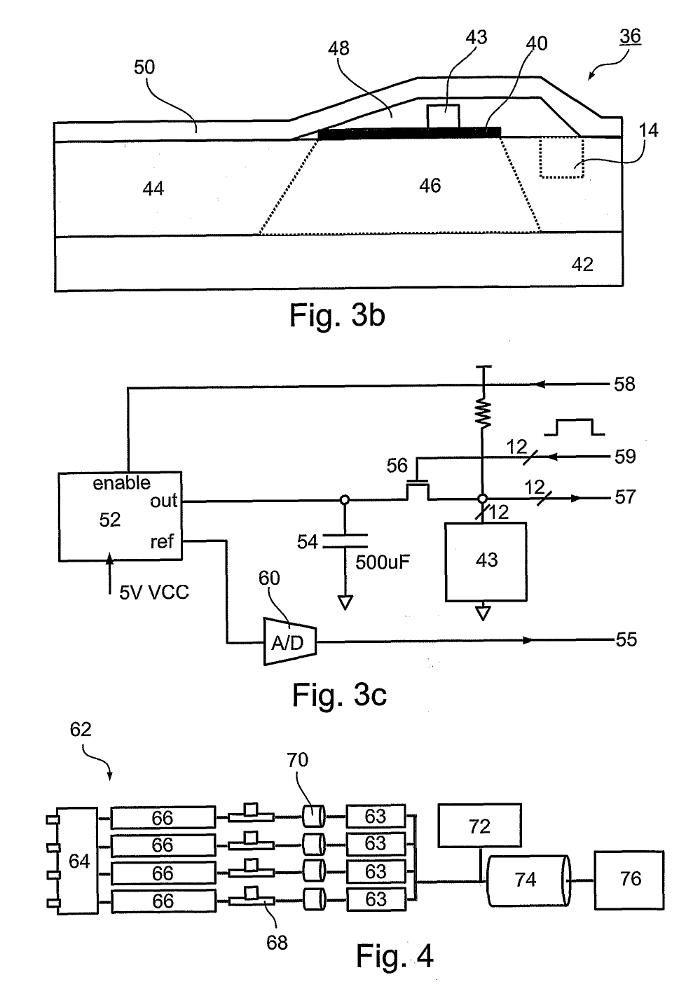

There are many techniques for actuating fluid transport in microchannels. Generally, a transport mechanism 62 is employed (see FIGS. 3a and 4, below). For example, in one embodiment transport mechanism 62 is capable of pumping or injecting sample fluid 15 through channels 14. Mechanism 62 can be placed on or in device 10 or not, depending on considerations such as costs, size the like.

Several examples of micro-pumps or micro-injectors which can be utilized in mechanism 62 are known in the art. Mechanism 62 preferably enables sample 15 delivery by applying a negative pressure to actuator port 34, channels 14 or reaction chambers 12, thereby delivering sample 15 from sample port 16 to reaction chambers 12.

As used herein “negative pressure” refers to a pressure value, which is smaller than a pressure value in a reference volume. For example, with respect to sample port 16, “negative pressure” refers to a pressure value which is smaller than the pressure value in sample port 16. The terms “negative pressure” and “under-pressure” are interchangeably used herein.

Reference is now made to FIG. 3a which is a schematic illustration of device 10 in the embodiment in transport mechanism 62 comprises a plurality of micro-pumps 36 which are capable of generating a negative pressure in chambers 12 with respect to the pressure in sample port 16 (i.e. the pressure in chambers 12 is lower than the pressure in sample port 16). According to a preferred embodiment of the present invention micro-pumps 36 are controlled by an electric circuitry (not shown, see FIG. 3c), through a plurality of electrical contact, designated 38 in FIG. 3a.

FIG. 3b is a schematic illustration of one of micro-pumps 36. Micro-pump 36 preferably comprises a substrate 42 (e.g., glass substrate) onto which a layer 44 having a vacuum chamber 38 therein is applied. A puncturable membrane 40, is deposited on vacuum chamber 46 thus buffering between vacuum chamber and an additional chamber 48 being in fluid communication with channel 14. Membrane 40 can be made form any suitable materials, such as, but not limited to, silicon-nitride. Additional chamber is sealed by a cover 50, which is preferably, but not obligatory transparent.

When membrane 40 is punctured, the pressure in channels 14 drops thereby actuating flow of sample fluid from sample port 16 to reaction chamber 12. The puncturing of membrane 40 is preferably by a heat shock which can be applied, for example, using a heater 43, controlled by the electronic circuitry (not shown see FIG. 3c) and positioned on or close to membrane 40.

Reference is now made to FIG. 3c, which is a schematic diagram exemplifying a preferred configuration of electronic circuitry 50 controlling micro-pump 36. It is to be understood that the electric configuration of FIG. 3c, as well as the accompanying description is not to be considered as limiting.

Hence, circuitry 50 can have Circuitry 50 includes a DC to DC switching converter 52 which is capable of charging a capacitor 54, having a capacitance of, for example, 500 μF, to a predetermined voltage of, e.g., 18 volts. A control line 58 may be connected to switching converter 52 for enabling or controlling the charging of capacitor 54. Heater 43 can be connected to capacitor 54 through a Metal Oxide Semiconductor (MOS) transistor 56. When a short regulated pulse is applied through line 59 to one of the gates transistor 56, the gate opens and capacitor 54 is discharged through heater 43 thereby initiating the heat shock which punctures membrane 40 as further detailed hereinabove. A typical resistance of heater 43 is about 2Ω, a typical activating current is about 9 A, and a typical a pulse duration is about 20 μs. Circuitry 50 may further comprise several feedback lines. One feedback line, designated 55 can be connected, e.g., via an analog to digital converter 60, to switching converter 52 and can be used for monitoring the status of capacitor 54, another feedback line, designated 57 can be connected to transistor 56 for acquiring an activation status of heater 43, hence to indirectly monitor whether or not membrane 40 is punctured.

FIG. 4 is a schematic illustration of transport mechanism 62 in the embodiment in which external pumping is utilized. For illustrative purposes only, FIG. 4 shows four pumping channels. It is to be understood that any number of pumping channels can be used.

According to a preferred embodiment of the present invention mechanism 62 comprises a pump interface 64 adapted to be connected to actuator port 34 or channels 14 and a vacuum chamber 74, interposed between pump interface 64 and a pumping device 76, and being in fluid communication therewith. In operational mode, pumping device 76 reduces the pressure in vacuum chamber 74, such that vacuum chamber 74 maintains a negative pressure environment. As a result, the pressure in interface 64 and actuator port 34 drops and actuates the flow of sample fluid 15 to reaction chamber 12.

Mechanism 62 may further comprise a pressure sensor 72 for monitoring the pressure at the inlet of vacuum chamber 74. Optionally and preferably, each pumping channel of mechanism 62 may further comprise a valve 63 capable of activating and deactivating the transport of sample fluid. Valve 63 is preferably a fast switching valve. A typical time delay of valve 63 is about 5 ms. The flow parameters (e.g., speed, volume) of sample fluid 15 in each pumping channel is preferably monitored using a flow sensor 66 and regulated using a tap 68. When sample fluid 15 is liquefied, the liquid may case damage to valve 63. According to a preferred embodiment of the present invention, mechanism 62 preferably comprises a filter 70 made of a hydrophobic material which prevents sample fluid 15 from arriving to valve 63. In the hydrophobic material of filter 70, cohesive forces between like molecules dominate over external forces existing between the molecules of the liquid and molecules of filter 70. The free surface of the liquid becomes film-like and the liquid is incapable of wetting filter 70 or penetrating therethrough.

The transport of sample fluid 15 may also be generated by electrical forces. When an uncharged particle (which may be, for example, a drop of sample fluid 15) is placed in a non-uniform electric field, it becomes polarized, i.e., acquires a non-zero electric dipole moment. The interaction between the electric dipole moment and the electric field results in net force acting on the fluid drop, which force is proportional to the electric dipole moment and the gradient of the electric field, and is commonly termed a dielectrophoretic force.

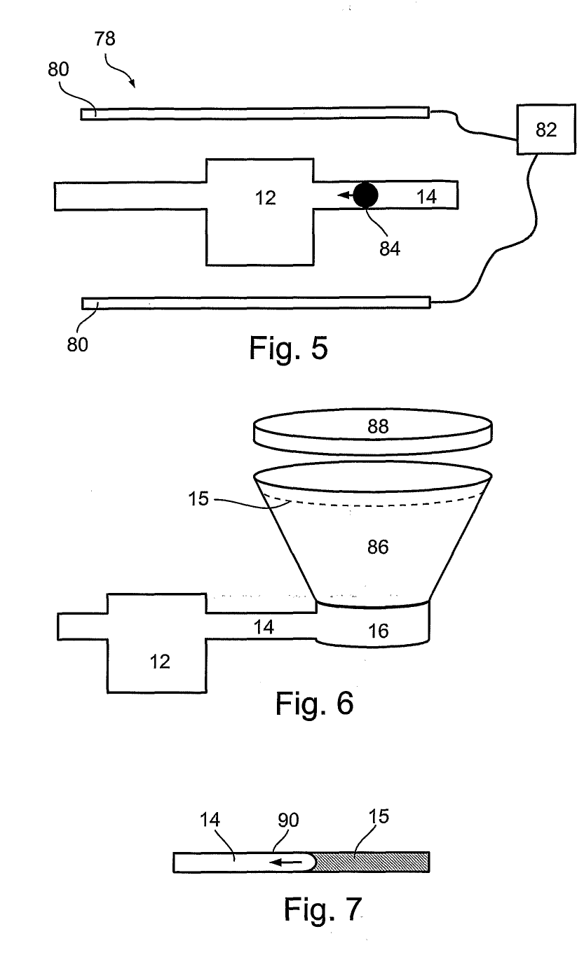

Reference is now made to FIG. 5, which is a simplified illustration of a portion of device 10 in which mechanism 62 comprises an electric field generator 78, according to a preferred embodiment of the present invention. Electric field generator 78 can be any device capable of generating a non-uniform electric field which induces polarization on molecules of sample fluid 15. A representative example include, without limitation, two plates 80 of variable conductivity connected to a voltage source 82. When a voltage is applied to plates 80, dielectrophoretic forces generated by the non-uniform electric field maneuver drop 84 of sample fluid 15 through the fluid channel.

The electric field is preferably designed and configured such that the dielectrophoretic forces direct drop 84 into reaction chambers 12. Alternatively, the electric field is can be designed and configured such that the dielectrophoretic forces direct drop 84 away from reaction chambers 12, for example, when it is desired to maneuver drop 84 from one chamber (e.g., a filled or partially filled chamber) to an empty chamber (e.g., an empty chamber). Still alternatively, the electric field is can be designed and configured such that the dielectrophoretic forces direct one portion of sample fluid 15 into reaction chambers 12 and another portion away from reaction chambers 12, all depending on the desired filling of device 10.

Reference is now made to FIG. 6 which is a simplified illustration of a portion of device 10 in an embodiment in which the transport of sample fluid 15 is generated by hydrostatic pressure. Hence, in this embodiment mechanism 62 preferably comprises a column 86 of sample fluid 15 connected to sample port 16. According to a preferred embodiment of the present invention the height of the column is selected such that the resulting hydrostatic pressure is sufficient for injecting sample fluid 15 into channels 14 and chambers 12. Optionally and preferably, column 86 may be supplemented by a pressing device 88 (e.g., a piston) for further increasing the pressure thereby to improve the flow of sample fluid 15 in channels 14.

An additional transport technique which is contemplated is transport via capillary action. A capillary action is a phenomenon in which adhesion forces between molecules of the fluid and molecules of solid cause the fluid to flow through a small diameter channel. Hence, referring to FIG. 7, according to a preferred embodiment of the present invention sample fluid 15 flows from sample port 16 into channels 14 via capillary forces generated between sample fluid 15 and walls 90 of channels 14.

As stated, sensors 18 may generate optical signals when exposed to the analyte(s) in sample fluid 15. This may be done by incorporating luminescent or fluorescent material in sensors 18. The present invention successfully provides a system 20 for detecting the analyte using optical signal.

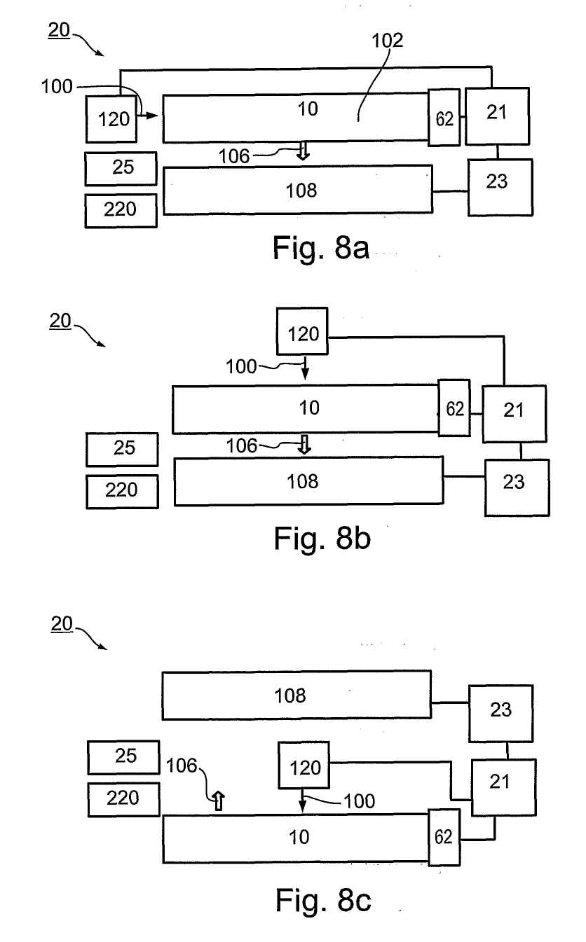

Reference is now made to FIGS. 8a-c, which are simplified illustrations of system 20. In its simplest configuration system 20 comprises a detecting device, e.g., device 10, and a light detector 108 for detecting an optical signal 106 generated by sensors 18 (not shown in FIGS. 8a-c). In the embodiment in which transport mechanism 62 is not an integral part of device 10, system 20 may further comprise transport mechanism 62. According to a preferred embodiment of the present invention system 20 further comprises a control unit 21 and a data processor 23. Control unit 21 sends control signals to components of system 20, for timing their operation. For example, control unit 21 may send activating and deactivating signals to light source 120 or mechanism 62. Data processor 23 serves for processing signals received from detector 108 and thus is in data communication therewith.

System 20 may further comprise a power source 220 for supplying energy thereto. Power source 220 can be fixed or portable, replaceable or rechargeable, integrated with or being an accessory to system 20. Examples of fixed power sources include, but are not limited too, a power source from a wall socket and a fixed voltage generator. Examples of a mobile power sources include, but are not limited too, an electrochemical cell (e.g., a battery) and a mobile a voltage generator.

When power source 220 is portable, it can be implemented in device 10, data processor 230, light detector 108 or any other component of system 20. In this embodiment, power source 220 can be, for example, a traditional secondary (rechargeable) battery, a double layer capacitor, an electrostatic capacitor, an electrochemical capacitor, a thin-film battery (e.g., a lithium cell), a microscopic battery and the like. The type and size of power source 220 as well as the amount of energy stored therein may vary, depending on the required power and, in some embodiments, on the component in which power source 220 is implemented. For example, when data processor 230 is a portable computer, power source 220 can be an internal battery of the portable computer.

Detector 108 receives optical signal 106 from sensors 18 and converts signal 106 into electronic signals (e.g., analog or preferably digital) which in turn can be received and analyzed, for example, by data processor 23. Detector 108 preferably detects optical signals 106 simultaneously from several reaction chambers. More preferably, detector 106 detects optical signals 106 simultaneously from all the reaction chambers.

Data processor 23 is preferably designed to include software for determining the presence, absence or concentration of the analyte in sample fluid 15. For example, data processor 23 can determine whether or not sample fluid 15 is toxic and send an appropriate sensible signal to the user which can monitor the sensible signal, e.g., using a display. Data processor 23 can also calculate the concentration of the analyte in sample fluid 15 and provide the user with the information desired.

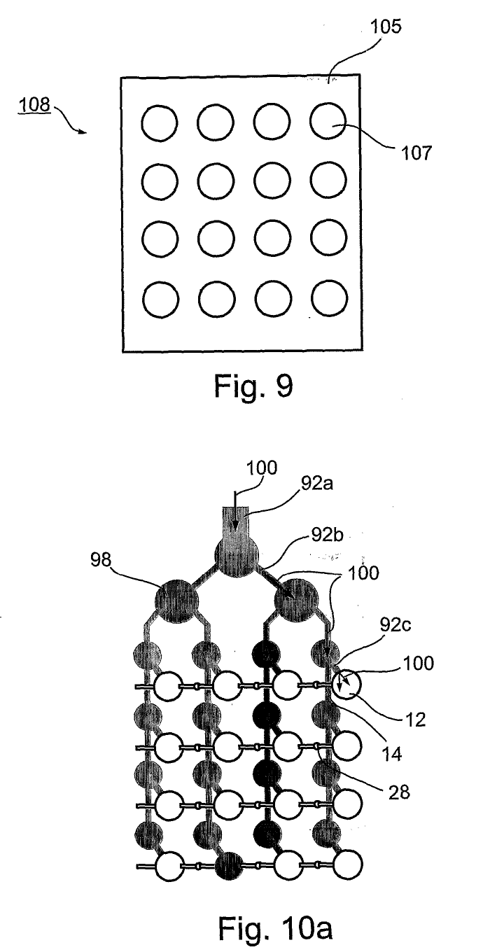

Reference is now made to FIG. 9, which is a simplified illustration of detector 108, according to a preferred embodiment of the present invention. Detector 108 preferably comprises a matrix 105 having a plurality of addressable elementary units 107, each being capable of converting light into electrical signal. Each elementary unit is allocated for a specific reaction chamber. When optical signal 106 originating from a particular reaction chamber impinges on matrix 105, the respective elementary unit generates a signal, which can then be analyzed by data processor 23. The signal generated by elementary units 107 preferably includes imagery information so as to allow attributing each signal to a respective reaction chamber, thereby providing an image thereof. Thus, according to a preferred embodiment of the present invention detector 108 is capable of providing an image of the sensors which generate optical signals 106.

Several types of elementary detection units are contemplated herein. For example, elementary units 107 can be positive-intrinsic-negative (PIN) photodiodes. A PIN photodiode is a device having a large, neutrally doped intrinsic region sandwiched between p-doped and n-doped semiconducting regions. A PIN diode exhibits an increase in electrical conductivity as a function of the intensity, wavelength and modulation rate of incident radiation. The avalanche photodiode, is preferably used in accordance with the present invention since it is capable of generating an amplified current by avalanche multiplication in which electrons, initially generated by the incident light, accelerate and collide with other electrons.

Detector 108, which incorporates PIN photodiodes or avalanche photodiodes enables accurate monitoring of intensity as well as the wavelength of optical signal 106.

According to an alternative embodiment, detector 108 employs a charge-coupled device (CCD), in which elementary units 107 are silicon chips. When light hits the silicon chip, electrons are released from the crystalline structure of the silicon and deposited into small units or wells. Once the image is captured, the electrons in the wells are sent into a recorder where they are counted.

In another embodiment, detector 108 comprises at least one photomultiplier. Typically, a photomultiplier is a vacuum tube including a photocathode which is capable of converting light into electrons, by virtue of the photoelectric effect, an electron multiplier and an anode. When light enters the photocathode, the photocathode electrons are emitted into the vacuum and then directed by a system of focusing electrode towards the electron multiplier. The electron multiplier is a string of successive electron absorbers with enhanced secondary emission hence multiply the numbers of electrons. The amplification of the electron multiplier can reach eight orders of magnitude. Once multiplied, the electrons are collected by the anode as an output signal. Because of the high secondary-emission multiplication, the photomultiplier provides extremely high sensitivity and low noise.

According to yet another alternative embodiment, detector 108 employs complementary metal oxide semiconductor (CMOS) technology. The advantage of using the CMOS technology is that the elementary units and various quantification parts can be integrated into a single device, which may be compact and simple to operate. Such CMOS are commercially available such as for example the ACS-1394 fire-wire camera based on the ACS-1024 CMOS Image Sensor manufactured by Photonics Vision Systems, or IBIS4 CMOS Image Sensor manufactured by Fill Factory http://www.fillfactory.com). Further description of a CMOS imaging sensor which can be used as detector 108, is provided in the Examples section which follows.

According to a preferred embodiment of the present invention system 20 further comprises at least one temperature control unit 25, for controlling the temperature of system 20. For example, temperature control unit 25, can monitor and adjust the temperature of device 10, detector 108 and/or light source 120 so as to optimize their operation. Temperature control unit 25 can be, for example, a thermoelectric device.

A thermoelectric device is a device that either converts heat directly into electricity or transform electrical energy into pumped thermal power for heating or cooling. Such a device is based on thermoelectric effects involving relations between the flow of heat and electricity through solid bodies. Generally, a thermoelectric device comprises at least one pair of dissimilar metals. When the device is used for cooling or heating, a potential difference is applied on the dissimilar metals and heat is pumped from one metal to the other. When the device is used for converting heat to electricity (e.g., for the purpose of monitoring the temperature of an object relative to a reference environment), the two metals are kept at different temperatures, and a potential difference is produced across.

Other temperature control units include, but are not limited to, liquid coolers, gas coolers, blowers and the like.

When sensors 18 include fluorescent material, optical signal 106 is generated in response to an excitation light 100, emitted by light source 120. Several configurations for positioning light source 120 are contemplated, depending on the relative angle between the detected portion of optical signal 106 and excitation light 100. For example, in one embodiment, illustrated in FIG. 8a, light source 120 is positioned on the side of device 10, such that the detected portion of optical signal 106 is substantially perpendicular to excitation light 100. In another embodiment, illustrated in FIG. 8b, light source 120 is positioned above or below device 10 in a manner such that device 10 is between light source 120 and light detector 108. In this embodiment, the detected portion of optical signal 106 is substantially parallel to excitation light 100. In an alternative embodiment, illustrated in FIG. 8c, light source 120 is positioned between device 10 and light detector 108. In this embodiment, the detected portion of optical signal 106 is substantially anti-parallel to excitation light 100. These embodiments are further detailed and exemplified hereinafter.

It is to be understood that, although FIGS. 8a-c show light detector 108 positioned below device 10, this configuration is not to be considered as limiting.

The low fluorescence quantum yield of presently available fluorescent materials requires a separation between the optical signal and the excitation light. According to a preferred embodiment of the present invention the separation of the excitation light from the optical signal can be spatial separation and/or in the spectral separation.

As used herein, the term “spatial separation” refers to confinement of light energy to propagate only in a predetermined volume, irrespective of its wavelength, and the term “spectral separation” refers to absorption or reflection of certain wavelengths and transmission of other wavelengths.

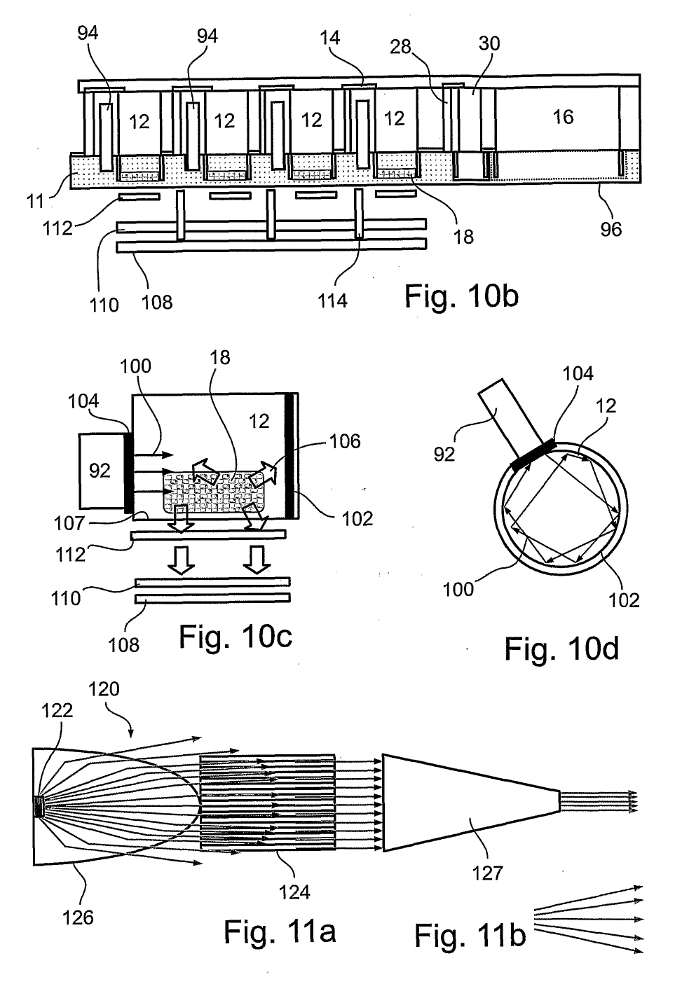

Reference is now made to FIG. 10a-d, which are simplified illustrations of device 10 in an embodiment in which spatial separation of the excitation light from the optical signal is employed. This embodiment is useful when the configuration of FIG. 8a is employed. Hence, according to the presently preferred embodiment of the invention device 10 comprises a plurality of waveguides 92 distributing excitation light 100 among chambers 12.

As the present embodiment relies upon the ability to transmit and emit light through a waveguide, a brief description of such technology is provided hereinbelow.

The technology to transmit and guide light rays through optical systems exploits a physical phenomenon known as total internal reflection, in which a light is confined within a material surrounded by other materials with lower refractive index. When a ray of light moves within a transparent substrate and strikes one of its internal surfaces at a certain angle, the ray of light can be either reflected from the surface or refracted out of the surface into the open air in contact with the substrate. The condition according to which the light is reflected or refracted is determined by Snell's law, which is a mathematical relation between the impinging angle, the refracting angle (in case in case of refraction) and the refractive indices of both the substrate and the air. Broadly speaking, depending on the wavelength of the light, for a sufficiently large impinging angle (also known as the critical angle) no refraction can occur and the energy of the light is trapped within the substrate. In other words, the light is reflected from the internal surface as if from a mirror. Under these conditions, total internal reflection is said to take place.

Many optical devices operate according to the total internal reflection phenomenon. One such optical device is the optical fiber. Optical fibers are transparent flexible rods of glass or plastic, basically composed of a core and cladding. The core is the inner part of the fiber, through which light is guided, while the cladding surrounds it completely. The refractive index of the core is higher than that of the cladding, so that light in the core impinging the boundary with the cladding at a critical angle is confined in the core by total internal reflection.

As stated, total internal reflection occurs only for light rays impinging the internal surface of the optical fiber with an angle which is larger than the critical angle. Thus, a calculation performed according to geometrical optics may provide the largest angle which is allowed for total internal reflection to take place. An important parameter of every optical fiber (or any other light transmitting optical device) is known as the “numerical aperture,” which is defined as the sine of the largest incident light ray angle that is successfully transmitted through the optical fiber, multiplied by the index of refraction of the medium from which the light ray enters the optical fiber.

Another optical device designed for guiding light is the graded-index optical fiber, in which the light ray is guided by refraction rather than by total internal reflection. In this optical fiber, the refractive index decreases gradually from the center outwards along the radial direction, and finally drops to the same value as the cladding at the edge of the core. As the refractive index does not change abruptly at the boundary between the core and the cladding, there is no total internal reflection. However, although no total internal reflection takes place, the refraction bends the guided light rays back into the center of the core while the light passes through layers with lower refractive indexes.

Optical fibers are available in various lengths and core-diameters. For large core diameters, glass optical fibers are known to be more brittle and fragile than plastic optical fiber.

Another type of optical device is based on photonic materials, where the light ray is confined within a band gap material surrounding the light ray. In this type of optical device, also known as a photonic material waveguide, the light is confined in the vicinity of low-index region. One example of a photonic material waveguide is a silica fiber having an array of small air holes throughout its length. This configuration is capable of providing lossless light transmitting, e.g., in either cylindrical or planar type waveguides.

Thus, according to a preferred embodiment of the present invention, each of waveguides 92 can be an optical fiber, a graded-index optical fiber a photonic material or any other optical device capable of transmitting light.

It is expected that during the life of this patent many relevant technologies for guiding light will be developed and the scope of the term waveguide is intended to include all such new technologies a priori.

Irrespectively of their type and operation principle, waveguides 92 can be integrated with or formed in body or substrate 11 of device 10. Alternatively, device 10 may be manufactured with a plurality of grooves 94 (see FIG. 10b) sizewise compatible with waveguides 92 such that waveguides 92 are inserted into grooves 94 prior to the excitation procedure. This embodiment is particularly useful when device 10 is made of a disposable material and it is desired to keep waveguides 92 for additional uses once device 10 is discarded.

As shown in FIG. 10a, waveguides 92 are preferably arranged in a multi-furcated arrangement (“a tree”), having a plurality of light splitting junctions 98, such that excitation light 100 enters through a single primary waveguide, designated 92a, and distributed by light splitting junctions 98 to secondary waveguides, designated 92b and 92c. Each light splitting junction 98 is preferably designed to satisfy the numerical apertures of its outgoing waveguides. Waveguides 92 may also be arranged in several multi-furcated trees, so that excitation light 100 can enter device 10 through several primary waveguides. This embodiment is useful when several excitation wavelengths are used, whereby each multi-furcated tree of waveguides is dedicated to a particular excitation wavelength.

In any event, according to a preferred embodiment of the present invention waveguides 92 distribute the light in a manner such that impingement of the excitation light on sensors 18 is maximized and impingement of excitation light 100 on a surface 96 of substrate 11 is minimized. Once the fluorescent material in sensors 18 is excited by light 100, an optical signal 106 is generated and can be detected, for example, using a light detector 108 position in the light path of signal 106, as further detailed hereinafter.

The minimization of impingement on surface 96 and the maximization of impingement on sensors 18 can be better understood from FIGS. 10c-d which illustrate a side view (FIG. 10c) and a top view (FIG. 10d) of one waveguide 92 guiding light 100 into chamber 12.

Referring to FIG. 10c, the minimization of the impingement of excitation light 100 on surface 96 can be achieved by imposing a predetermined propagation direction on light 100. More specifically, according to a preferred embodiment of the present invention, when exiting waveguide 92 and entering chamber 12, light 100 propagates a direction which is substantially parallel to surface 96.

Referring to FIG. 10d, the maximization of impingement of light 100 on sensors 18 can be achieved by allowing light 100 to exit waveguide in a plurality of co-planar direction, each being parallel to surface 96. A predetermined propagation direction can be imposed on light 100 either directly by waveguide 92 or by one or more additional optical elements 104, e.g., a diffraction grating, a reflection grating, a mini-prism and the like. Optionally and preferably chamber 12 my comprise a reflective coat 102, covering the walls of chamber 12, so as to reflect light 100 hence to further increase the impingement of light 100 of sensors 18. As further detailed hereinunder and in the Examples section that follows, sensors 18 may be biological sensors. In this embodiments coat 102 is preferably made of a biocompatible material.

Thus, excitation light 100 is constrained to propagate in a predetermined direction while an optical signal 106, generated by sensors 18 in response to light 100, is allowed to propagate in all directions. According to a preferred embodiment of the present invention at least one side of chamber 12 (e.g., a bottom side 107) is not coated by coat 102. One of ordinary skill in the art would appreciate that with such configuration, optical signal 106 can be detected by detector 108 without being screened by excitation light 100 which is substantially confined in chamber 12.

It is appreciated, however, that light 100 can be diverted, for example, when light 100 is not absorbed by sensors 18 but rather being scattered to a different direction. Thus, according to a preferred embodiment of the present invention, the aforementioned spatial separation is combined with spectral separation. For example, an emission filter 110 can be positioned in the light path of optical signal 106 so as to prevent diverted rays of excitation light 100 from arriving to detector 108. Emission filter 110 preferably allows transmission of optical signal 106 substantially without loses. Additionally or alternatively, coat 102 can be a selective coat, capable of selectively reflecting light of a particular wavelength. In this embodiment, coat 102 may cover also bottom side 107 of chamber 12 so that optical signal 106 is transmitted therethrough and light 100 is reflected thereby.

According to a preferred embodiment of the present invention, optical signals generated in different reaction chambers are spatially separated so as to prevent cross talks between the different optical signals. This can be done, for example, by positioning an optical focusing device 112 (e.g., a microlens) in the light path of optical signal 106 so as to focus signal 106 on detector 108. Alternatively device 112 can be positioned so as to collimate signal 106 to a predetermined direction. In this embodiment, the optical signals of different chambers are preferably collimated to propagate in parallel directions thereby preventing cross talks therebetween. Optionally, a plurality of optical separations 114 can be positioned between different optical signals so as confined each optical signal not to cross the light path of the other optical signals. Optical separation 114 can be, for example, a reflector to minimize losses.

Reference is now made to FIG. 11a, which is a simplified illustration of a side view of light source 120, in the embodiment in which waveguides 92 are employed. According to a preferred embodiment of the present invention light source 120 comprises a light emitting device 122, a collimator 124 and a light coupler 127. Light emitting device 122 can be, for example, a light emitting diode (LED), covered by a collimating lens 126, capable of partially collimating the light emitted by the LED. Typically, lens 126 has a diameter of about 5 millimeters and is capable of providing a beam having a divergence of about 15°. Collimator 124 serves for further collimating light 100 and light coupler 127 serves for reducing the diameter of the light beam so as to facilitate coupling of light 100 into waveguide 92. Light coupler 127 can be any known device for coupling a light into a waveguide in a manner that the impinging angle of the light on the waveguide satisfies its numerical aperture. One such light coupler which is commercially available is known as a “pigtail.”

FIG. 11b, schematically illustrate a top view the light beam outputted by light source 120. As shown in FIG. 11b, the light beam propagate in a plurality of co-planar direction thus maximizing the impingement of light 100 on sensors 18 as further detailed hereinabove.

Typically but not obligatory, light emitting device 122 emits blue light (e.g., wavelength of about 470 μm) at an optical power of about 5 mW. Typical dimensions of light emitting device 122 are about 5 mm in width and about 10 mm in length.

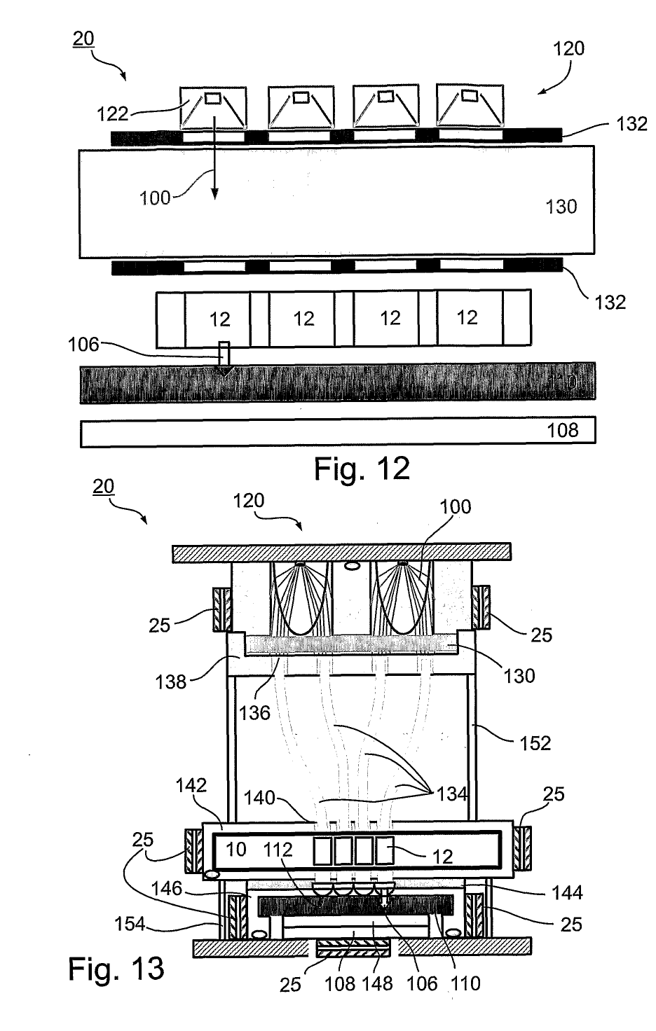

Reference is now made to FIG. 12 which is a simplified illustration of system 20 in an embodiment in which device 10 is positioned between light source 120 and detector 108. Thus, in this embodiment, the detected portion of optical signal 106 is substantially parallel to excitation light 100.

It is recognized that when optical signal 106 is parallel to excitation light 100, a spectral separation between optical signal 106 and excitation light 100 is required. Thus, system 20 preferably comprises one or more selective filters for selectively allowing transmission of light having predetermined wavelength. One such selective filter is preferably an excitation filter 130 which allows transmission of excitation light 100 and substantially prevents transmission of light having different wavelengths. Another such selective filter is the aforementioned emission filter 110 which allows transmission of optical signal 106 and substantially prevents transmission of light having different wavelengths.

To facilitate substantially simultaneous excitation of the sensors in all or at least a portion of chambers 12, light source 120 preferably comprises a plurality of light emitting devices, which can be, for example, similar to the aforementioned light emitting device 122. Optionally and preferably, system 20 comprises one or more separators 132 for substantially preventing cross talks between different excitation light rays.

To provide a better separation between excitation light rays emitted by different light emitting devices, system 20 can employ an arrangement of optical fibers, as further detailed hereinbelow.

Reference is now made to FIG. 13, which is a schematic illustration of system 20 in an embodiment in which a plurality of external optical fibers are employed. Generally, in this embodiment system 20 comprises a first housing 138 holding devices 122 and excitation filter 130, a second housing 142 for holding device 10 and a third housing 146 for holding detector 108 and emission filter 110. First hosing 138 is preferably connected to second housing 142 by supporting legs 152, and second housing 142 is preferably connected to third housing by supporting legs 154.

According to a preferred embodiment of the present invention system 20 comprises a plurality of optical fibers 134 which deliver excitation light 100 from emitting devices 122 to device 10. Preferably, as shown in FIG. 13, each optical fiber delivers excitation light 100 to one of chambers 12. Alternatively, one optical fiber can deliver light 100 to more than one chamber. A first end of each of optical fibers 134 is preferably connected via a groove 136 to housing 138 and a second end thereof is preferably connected, via a groove 140 to second housing 142.

First housing 138 and second housing 142 are preferably made of a thermally conductive material, so as to allow temperature control unit 25 to monitor and control the temperatures thereof. Optionally, system 20 can comprise one or more thermistors being in thermal communication with first 138 and/or second 142 housings, for sensing the temperatures.

Second housing 142 is preferably thermally isolated from detector 108. This can be done, for example, using one or more thermal isolators 144 positioned adjacently to second housing 142 and/or third housing 146.

Optionally an additional filter, an infrared filter 148, can be positioned in the light path of optical signal 106 so as to filter out infrared radiation which may be generated by third housing 146, when its temperature is rising.

As stated, optical signals generated in different reaction chambers can be spatially separated, for example, using an optical focusing device 112, so as to prevent cross talks between the different optical signals. Representative examples of device 112 include without limitation, a lens, a plurality of lenses (e.g., a micro-lens matrix) and a video lens. Device 112 can be positioned on second 142 or third housing 146. Similarly to the above description, device 112 can either focus optical signal 106 on detector 108 or collimate optical signal 106 such that optical signals of different chambers propagate substantially in parallel directions.

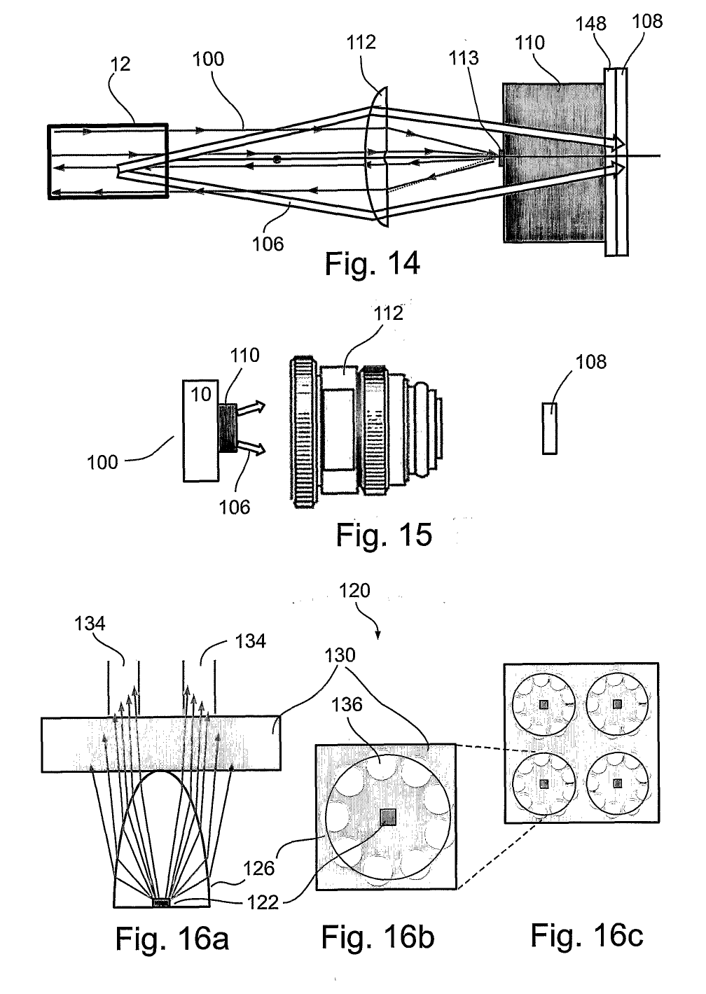

Optical focusing device 112 can also be used for further separation of excitation light 100 from optical signal 106. This can be better understood from FIG. 14, which is a simplified illustration of the light path of excitation light 100 once entering chamber 12. FIG. 14 is rotated anticlockwise by 90° relative to FIG. 13.

Due to the use of optical fibers 134, the rays of light 100 are substantially parallel. When light 100 enters chamber 12 it can (i) absorbed by sensors 18 which in response emits optical signal 106; (ii) scatter off sensors 18 and continue to propagate in a diverted direction; or (iii) continue to propagate in its original direction without interacting sensors 18. Focusing device 112 is preferably oriented in a manner such that the parallel, non-interacting, light rays are focused by focusing device 112 to its focal point. According to a preferred embodiment of the present invention an opaque object or a reflector 113 can is positioned in the focal point of focusing device 112 so as to absorb or reflect light 100 hence to prevent it from arriving to detector 108. Reflector 113 is preferably sufficiently small so as not to absorb or reflect off-focal rays. Unlike light 100, optical signals 106 are emitted and propagated in a plurality of directions, so that only a small portion of optical signals 106 is focused to the focal point of focusing device 112. Being sufficiently small, the effect of reflector 113 is negligible for optical signals 106. On the other hand, excitation light rays which are scatter off sensors 18 without being absorbed thereby arrive to focusing device 112 in a direction which may be not parallel to its focal axis. Such non-parallel rays, however, are absorbed by emission filter 110.

Reference is now made to FIG. 15 which illustrates focusing device 112 in the embodiment in which focusing device 112 is a video-lens. The advantage of using a video-lens is that this device is capable of simultaneously projecting optical signals 106 from many reaction chambers to detector 108, substantially without cross-talks. In addition, a video-lens having a low focal number is capable of collecting a significant part of optical signal 106 with minimal loses.

Reference is now made to FIGS. 16a-c, which are schematic illustrations of light source 120 in the embodiment of system 20 in which external optical fibers 134 are employed (see FIG. 13). Hence, light source 120 preferably comprises a plurality of light emitting devices 122 covered by collimating lens 126 and arranged in a manner that light emitted by each one of devices 122 enters on one or more of optical fibers 134. For example, a plurality of grooves 136 can be circularly arranged in front of a collimating lens of a single light emitting device, such that light rays having a predetermined impinging angle enter the optical fibers. One of ordinary skill in the art will appreciate that the circular arrangement of grooves 136 ensures that each optical fiber is impinged by the light substantially at the same angle. Referring to FIG. 16c, when a plurality of light emitting devices 122 is used, each device can provide excitation light to many optical fibers hence also to many reaction chambers. For example, in the embodiment shown in FIG. 16c, there are four light emitting devices, each providing excitation light to nine optical fibers, hence to nine reaction chambers.

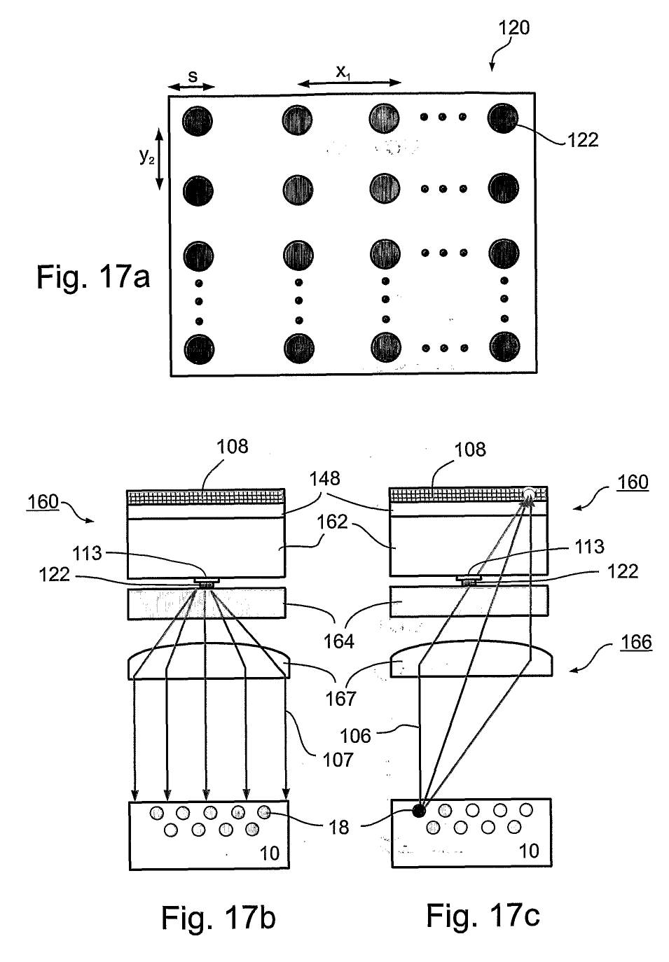

Reference is now made to FIGS. 17a-c which are simplified illustrations of system 20 in the embodiment in which light source 120 is positioned between device 10 and light detector 108. In this embodiment, system 20 preferably comprises device 10 and an apparatus 160 for imaging the pattern of optical signals 106. Apparatus 160 comprises detector 108, an optical element 166 which may be, for example, a plurality of lenses 167 and light source 120. Lenses 167 are preferably arranged in an arrangement which is compatible with the arrangement of chambers 12 in device 10, such that each lens is allocated to a predetermined number of chambers (e.g., one lens per chamber).

Referring to FIG. 17a, light source 120 can be an arrangement of light emitting devices 122, which is preferably compatible with the arrangement of lenses 167 such that each light emitting device is allocated to a predetermined number of lenses (e.g., one light emitting device per lens). Shown in FIG. 17a is a rectangular arrangement of light emitting devices 122, in which the distance between two adjacent light emitting devices is x₁ in one direction (say, the “x” direction) and y₁ in the orthogonal direction (say, the “y” direction). Typical value for both x₁ and y₁ is a few millimeters, for example, 1 millimeter. x₁ can be equal to, or different from y₁, depending on the desired geometrical arrangement, for example, the density of light emitting devices 122 in the respective direction. The transverse size of each light emitting device, designated s in FIG. 17a, is typically from about 10 μm to about 20 μm. Light emitting devices 122 can be activated simultaneously or independently.

FIGS. 17b-c show one light emitting device and one lens respectively designated by numerals 122 and 167. Detector 108 is preferably connected to a first substrate 162, which may be, for example, a glass substrate coated so as to prevent randomly reflected excitation rays from penetrating therethrough. In embodiments in which infrared filter 148 is employed, infrared filter 148 is preferably formed on first substrate 162 and detector 108 is connected to infrared filter 148. Light emitting device 122 is preferably connected to a second substrate 164, which can be, for example, a sapphire substrate.

According to a preferred embodiment of the present invention light emitting device is configured to generate excitation light 100 in a direction other than a direction of detector 108. This can be done, for example, by positioning opaque object or reflector 113 adjacently to light emitting device 122, between light emitting device 122 and detector 108 thereby to prevent light 100 from impinging on detector 108. Reflector 113 can also have a non planar shape (e.g., parabolic or hyperbolic shape) so as to increase the amount of excitation light propagating in the direction of device 10.

FIG. 17b show the light path of excitation light 100. As shown, light emitting device 122 is preferably positioned at the focal point of lens 167, so that excitation light 100 is collimated by lens 167, and impinges on sensors 18 of device 10 in a form of a collimated beam.

FIG. 17c show the light path of optical signals 106, emitted by sensors 18. According to a preferred embodiment of the present invention, lens 167 is positioned in a manner such that optical signals 106 are focused by lens 167 to impinge on detector 108. This can be achieved by positioning lens 167 half way between detector 108 and device 10, at two focal distances therefrom. Only collimated light rays have a light path which goes through the focal point of lens 167. Thus, being emitted at a plurality of directions, a large portion of optical signals 106 arriving at lens 167 is not collimated, and therefore is not affected by reflector 113.

The above selection of two focal distances between lens 167 and device 10 on the one side, and between lens 167 and detector 108 on the other side, ensures mapping of sensors 18 or chambers 12 on detector 108. More specifically each one of reaction chamber 12 is represented by an addressable region on detector 108. When sensors of a particular reaction chamber emit optical signal 106 detector 108 detects this signal at the respective addressable region. Thus an image the emitting sensors is formed on detector 108. Knowing the reactivity properties of the sensors of the respective reaction chamber in device 10, the image can be used, for example, by data processor 23 (not shown, see FIG. 8c), for determining the presence and/or concentration of the analyte(s) with which the sensors react.

As stated, the sensors which are employed by the present invention are capable of generating a detectable signal when exposed to the at least one analyte in the sample. According to a preferred embodiment of the present invention sensors 18 are biological sensors. Many biological sensors are contemplated. Preferably the biological sensors are made of a biological material (e.g., cell population) capable of producing a material when exposed to the analyte. Representative examples of the produced material include, without limitation, a bioluminescent material, a phosphorescent material and a fluorescent material. Alternatively, the bilogical sensors can produce a material which is capable of altering the electrostatic characteristic of the sample.

Although numerous examples of biological sensors exist in the art, these are limited by instability of the biological component, irreversibility, costs of production and limited ability to identify broad range of analytes.

To overcome such limitations, the present inventors have devised and constructed a reporter-expressing cell population which is composed of discrete subpopulations each capable of expressing the reporter in response to a different analyte or groups of analytes. When exposed to an analyte, the various subpopulations produce a specific expression pattern which forms a signature profile specific to the analyte present in the sample. To enable such analyte specific expression, the present inventors carefully selected a group of promoters which can be activated by different analytes from a number of promoter libraries. It is postulated herein that by utilizing a broad range of physiologically-responsive promoters, one increases an ability of a cell population transformed with reporter constructs containing such promoters to uniquely respond (via unique reporter expression patterns) to each of a broad range of analytes.

Thus, according to a further aspect of the present invention there is provided a population of cells which can be utilized as biological sensors. The population of cells is composed of at least two subpopulations of cells. A first such subpopulation includes a first reporter expression construct which is capable of reporter expression when the cells of this subpopulation are exposed to a first analyte. A second subpopulation of cells includes a second reporter expression construct which is capable of reporter expression when the cells of the second subpopulation are exposed to a second analyte.

As used herein “population of cells” refers to prokaryotic or eukaryotic cells which can be genetically modified (in a transient or stable manner) to express exogenous polynucleotides.

Examples of prokaryotic cells which can be used in accordance with this embodiment include but are not limited to bacterial cells, such as Pseudomonas, Bacillus, Bacteriodes, Vibrio, Yersinia, Clostridium, Mycobacterium, Mycoplasma, Coryynebacterium, Escherichia, Salmonella, Shigella, Rhodococcus, Methanococcus, Micrococcus, Arthrobacter, Listeria, Klebsiella, Aeromonas, Streptomyces and Xanthomonas.

Examples of eukaryotic cells which can be used in accordance with the present embodiment include but are not limited to cell-lines, primary cultures or permanent cell cultures of fungal cells such as Aspergillus niger and Ustilago maydis [Regenfelder, E. et al. (1997) EMBO J. 16:1934-[942], yeast cells (see U.S. Pat. Nos. 5,691,188, 5,482,835), such as Saccharomyces, Pichia, Zygosaccharomyces, Trichoderma, Candida, and Hansenula, plant cells, insect cells, nematoda cells such as c. elegans, invertebrate cells, vetebrate cells and mammalian cells such as fibroblasts, epithelial cells, endothelial cells, lymphoid cells, neuronal cells and the like. Cells are commercially available from the American Type Culture Co. (Rockville, Md.).

As mentioned hereinabove, the population of cells preferably includes at least two subpopulations of cells. However, it is appreciated that the more subpopulations included in the cell population the higher the chances of such a cell population to accurately identify analytes present in a sample exposed thereto.

As mentioned hereinabove, each subpopulation of cells includes a reporter expression construct, which expresses a detectable reporter molecule when the cell is exposed to an analyte.

As used herein “reporter expression construct” refers to a vector which includes a polynucleotide sequence encoding a reporter. The reporter expression construct is preferably designed to randomly integrate into the genome of the cell, such that expression of the reporter polypeptide is governed by an endogenous regulatory element which is inducible by an analyte.

According to a preferred embodiment of the present invention, the polynucleotide sequence is positioned in the construct under the transcriptional control of at least one cis-regulatory element suitable for directing transcription in the subpopulation of cells upon exposure to an analyte.

As used herein a “cis acting regulatory element” refers to a naturally occurring or artificial polynucleotide sequence, which binds a trans acting regulator and regulates the transcription of a coding sequence located down-stream thereto. For example, a transcriptional regulatory element can be at least a part of a promoter sequence which is activated by a specific transcriptional regulator or it can be an enhancer which can be adjacent or distant to a promoter sequence and which functions in up regulating the transcription therefrom.

It will be appreciated that the cis-acting regulatory element of the presently preferred embodiment of the invention may be stress regulated (e.g., stress-regulated promoter), which is essentially activated in response to cellular stress produced by exposure of the cell to, for example, chemicals, environmental pollutants, heavy metals, changes in temperature, changes in pH, as well as agents producing oxidative damage, DNA damage, anaerobiosis and changes in nitrate availability or pathogenesis.

Examples of promoters which are preferably used in accordance with the presently preferred embodiment of the invention include, but are not limited to, MipA, LacZ, GrpE, Fiu, MalPQ, oraA, nhoA, recA, otsAB and yciD.

A cis acting regulatory element can also be a translational regulatory sequence element in which case such a sequence can bind a translational regulator, which up regulates translation.

The term “expression” refers to the biosynthesis of a gene product. For example, in the case of the reporter polypeptide, expression involves the transcription of the reporter gene into messenger RNA (mRNA) and the translation of the mRNA into one or more polypeptides.

As used herein “reporter polypeptide” refers to a polypeptide gene product, which, can be quantitated either directly or indirectly. For example, a reporter polypeptide can be an enzyme which when in the presence of a suitable substrate generates chromogenic products. Such enzymes include but are not limited to alkaline phosphatase, β-galactosidase, β-D-glucoronidase (GUS), luciferase and the like. A reporter polypeptide can also be a fluorescer such as the polypeptides belonging to the green fluorescent protein family including the green fluorescent protein, the yellow fluorescent protein, the cyan fluorescent protein and the red fluorescent protein as well as their enhanced derivatives. In such a case, the reporter polypeptide can be quantified via its fluorescence, which is generated upon the application of a suitable excitatory light. Alternatively, a polypeptide label can be an epitope tag, a fairly unique polypeptide sequence to which a specific antibody can bind without substantially cross reacting with other cellular epitopes. Such epitope tags include a Myc tag, a Flag tag, a His tag, a Leucine tag, an IgG tag, a streptavidin tag and the like. Further detail of reporter polypeptides can be found in Misawa et al. (2000) PNAS 97:3062-3066.

It will be appreciated that in certain aspects of the present invention the reporter expression construct may be expressed in response to a growth condition. Examples of such conditions include, but are not limited to temperature, humidity, atmospheric pressure, contact surfaces, radiation exposure (such as, y-radiation, UV radiation, X-radiation).

As mentioned hereinabove, each reporter expression construct is expressed in a subpopulation of cells upon exposure to a distinct analyte or groups of analytes. It will be appreciated however, that since several unrelated analytes can lead to the same effect on a cell, an expression construct can also be expressed albeit at lower efficiency upon exposure to other analytes. Such partial overlap between the different reporter expression constructs is desirable since it will increase the detection range of the population to thereby enable identification of numerous analytes even at low concentration levels. For example, if a first analyte induces reporter expression from one subpopulation it may be difficult to distinguish it from a second unrelated analyte which also induces expression in the same subpopulation. However, if several cell subpopulations are induced by a first analyte (each subpopulation expressing a unique level of the reporter) the likelihood that the same subpopulations will also react with the same expression pattern upon exposure to a second analyte is remote.

Dependent on the host cell used, the reporter expression construct can include additional elements. For example, polyadenylation sequences can also be added to the reporter expression construct in order to increase the translation efficiency of a reporter polypeptide expressed from the expression construct of the present embodiment. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Suitable termination and polyadenylation signals include, without limitation, those derived from SV40.

In addition to the elements already described, the expression construct may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The construct may or may not include a eukaryotic replicon. If a eukaryotic replicon is present, then the vector is amplifiable in eukaryotic cells using the appropriate selectable marker. If the construct does not comprise a eukaryotic replicon, no episomal amplification is possible. Instead, the recombinant DNA integrates into the genome of the engineered cell, where the promoter directs expression of the desired nucleic acid.

The reporter expression construct can be introduced into the cell using a variety of molecular and biochemical methods known in the art. Examples include, but are not limited to, transfection, conjugation, electroporation, calcium phosphate-precipitation, direct microinjection, liposome fusion, viral infection and the like. Selection of a suitable introduction method is dependent upon the host cell and the type of construct used.

Since the response of each subpopulation of the cell population of the presently preferred embodiment of the invention to an analyte needs to be assessed independently in order to generate a signature expression pattern, each cell of each subpopulation is preferably tagged with a distinct tag unique to the subpopulation. The tag may be for example, a fluorophoric or chromophoric dye compound which may be detected using a microscope. Such dyes are commercially available such as from Molecular Probes (Eugene, Oreg., USA). Alternatively, cells can be naturally fluorescing or genetically engineered to fluoresce. Molecular tags can also be used. Such tags may be detected by amplification methods, such as PCR.

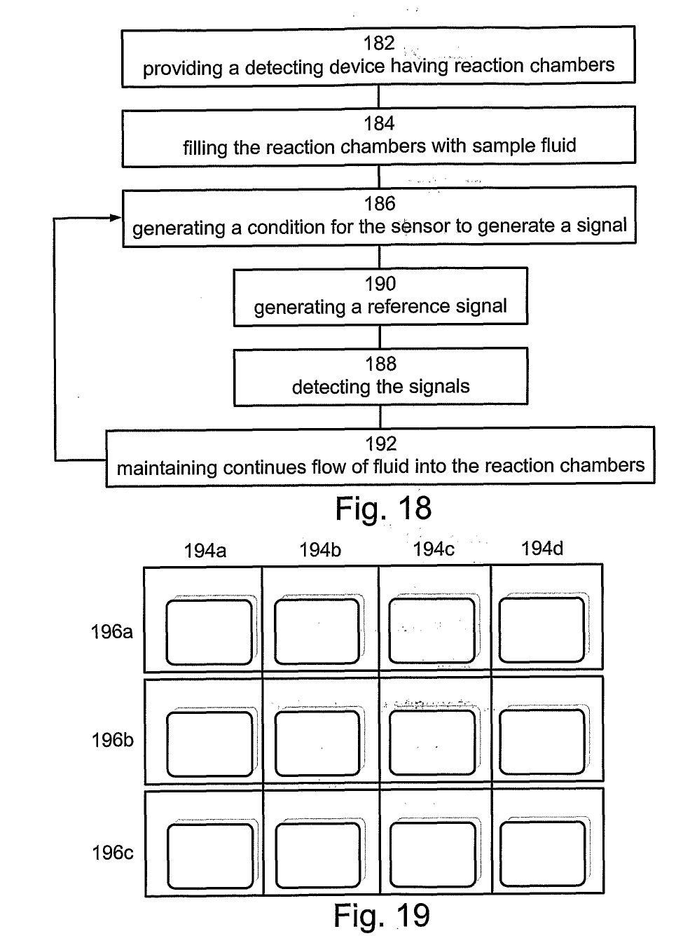

According to yet another aspect of the present invention there is provided a method of detecting analytes in a sample fluid, the method comprises the following method steps which are illustrated in the flowchart diagram of FIG. 18.

In a first step of the method, designated by Block 182 a detecting device, e.g., device 10 is provided. In a second step, designated by Block 184, a portion or all of the reaction chambers of the detecting device are filled with the sample fluid. The filling can be done by any of the aforementioned transport techniques, e.g., pumping, dielectrophoretic forces, capillary forces, injection and the like. According to a preferred embodiment of the present invention different portions of the reaction chambers of the detecting device can be contain different sensors. For example, when the sensors are cell population, different subpopulations can be placed in different reaction chambers. In addition, several reaction chambers can include only nutritious material for the subpopulations, so as to allow assessment of the contribution of the nutritious material to the detected signals. Still in addition, several reaction chamber may not contain sensors at all, thereby serving as a control group.

According to a preferred embodiment of the present invention different portions of the reaction chambers can be filled with different fluids, in any combination with the different sensors, so as to allow each sensor to be exposed to a plurality of fluids and each fluid to be sensed by a plurality of sensors. This embodiment is particularly useful for conducting complicated assays, as further detailed hereinunder and exemplified in the Examples section that follows.

In a third step of the method, designated by Block 186, the sensors generate the detectable signal. This can be done, for example, in response to irradiation of the device by excitation light, as further detailed hereinabove. In a fourth step, designated by Block 188, the signal is detected, for example, using a planar detector (e.g., detector 108).

According to a preferred embodiment of the present invention the method further comprises maintaining continues flow of the sample fluid in the channels so as continuously replace the sample fluid the reaction chambers. This step is designated by Block 192 in FIG. 18. To facilitate continuous flow, the device preferably comprises a draining system (e.g., output buffer 32, see FIG. 2c) for discarding excess fluids therefrom. Continues flow of sample fluid is advantageous for online monitoring and detection of the sample fluid. Thus, according to a preferred embodiment of the present invention the detection of the generated signals is done substantially in real time.

In an online measurement, it is often desired to have an indication of the general state of the detecting device. The electronic circuitries of the device (e.g. the aforementioned circuitries for controlling the transport mechanism, temperature, light source, detector, etc.) can be monitored substantially in real time by incorporating appropriate feedback lines therein. In addition, several sensors of the preferably generate a reference signal at all times, so as to provide indication of their operation. This embodiment is represented in FIG. 18 by Block 190.

When the sensors of the detecting device are biological sensors (e.g., live cells, such as, but not limited to, the aforementioned reporter-expressing cell population), the viability thereof can be monitored by incorporating in a few reaction chambers, a material (e.g., a biological material) which generate a detectable reference signal at all times. The reference signal can be optical, electrical electrochemical or any other signal.

The present embodiment has several advantages. First, the reference signal indicates viability of the detecting device. For example, lack of reference signal can indicate that the cell is dead or significantly damaged.

Second, when the sample is highly toxic, the biological sensors may fail to produce the detectable signal, for example, when the biological sensors are killed by toxic substances in the sample. On the other hand, highly toxic sample may also kill the biological material generating the reference signal. Thus, in this case a cessation of the reference signal indicates a highly toxic sample.

Third, the reference signal can serve as a sensor for abnormal state of the sample. For example, when the reference signal is generated by live cells, an abnormal sample state (e.g., abnormal temperature, abnormal pH level, etc.) may cause a stress to the live cells resulting in a decrease of their ability to generate normal reference signal. A reference signal which is below a predetermined level therefore indicates an abnormal sample state.

Thus, the combination of the reference signal and the sensors of the device allows an efficient detection of the analyte in the sample. According to a preferred embodiment of the present invention, the presence, concentration and/or type of the analyte can be determined. More specifically, the presence of analytes in the sample can be determined by detecting a change in signal reading or a change the rate of change of signal readings (both signal in response to the analyte and reference signals); the concentration of the analyte in the sample is determined by the absolute value of the detected signal; and the type of analyte is determined from the imaging information provided by the detector.

As stated, different portions of the reaction chambers of the detecting device can be contain different sensors and/or be filled with different fluids, in any combination with the different sensors. For example, in the embodiment in which the reaction chambers and the fluid channels are arranged in sequential arrays (see FIGS. 2a-b), each array can be allocated for a different sample fluid, while each reaction chamber in a particular array contain a different sensor. Thus, when a particular sample fluid is transported into the chambers of its respective array, different signals are generated in different reaction chambers, thus enable multiplexing.

Reference is now made to FIG. 19, which is a schematic illustration of a logical and physical division of the detecting device, according to the presently preferred embodiment of the invention. As show in FIG. 19, the detecting device can be divided into rows and columns. FIG. 19 exemplify four columns, designated 194a, 194b, 194c and 194d, and three rows, designated 196a, 196b and 196c. The reaction chambers of each column can be in fluid communication thereamongst, so as to allow filling al the reaction chambers of a particular column with the same sample fluid.

The columns can be physically divided so as to prevent sample fluids from flowing across a row of reaction chambers. Each row can be allocated to a different functionality. For example, row 196a can be allocated for signal stability testing, row 196b can be allocated for detecting unknown analytes and row 198c can be allocated for detecting known analytes.

Different columns can be allocated for different sample fluid or the same sample fluid as desired. For example, prior to the transport of the sample fluid into one column, the sample fluid can be purified, e.g., using an activated filter, thus to serve as a control to another column in which no purification was employed.

According to still another aspect of the present invention there is provided a method of dehydrating a biological material. The method can be used for example, when for placing biological sensors, e.g., the aforementioned cell population, in the reaction chambers of device 10, in a vacuum preservation manner. It is appreciated that when the cell population is dehydrated and kept in vacuum, the cell population can be in dormant state for a prolonged period of time, until device 10 becomes operative.

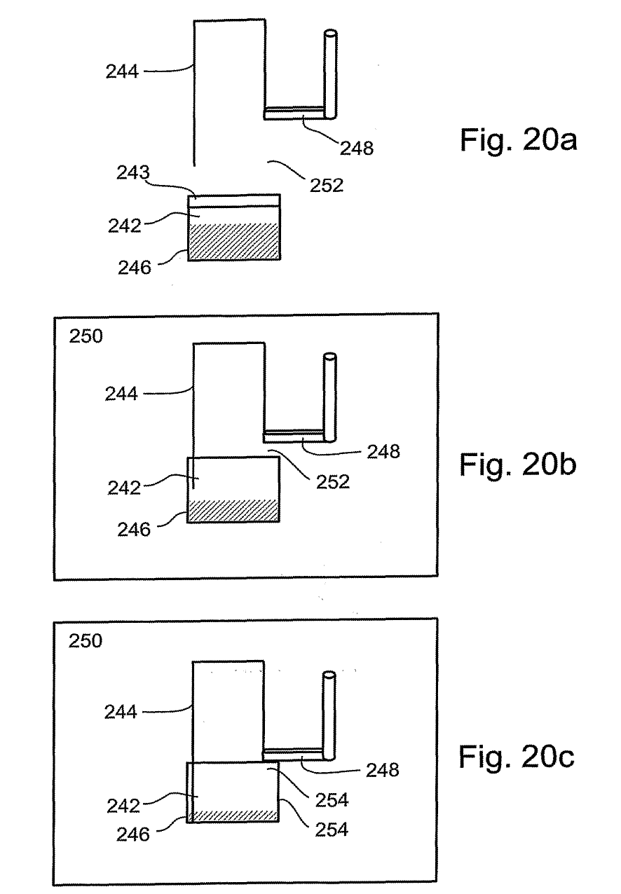

The method according to the presently preferred embodiment of the invention comprises the following method steps which are illustrated in FIGS. 20a-c. In a first step of the method, illustrated in FIG. 20a, a first set 242 and a second set 244 of chambers are provided. First set 242 preferably contains a biological material 246 and second set 244 preferably has one or more fluid channel 248 formed therein or attached thereto. It is to be understood that although each of first set 242 and second set 244 are represented in FIG. 20a-c a single chamber, any number of chambers can be used, in any arrangement, depending on the application for which biological material 246 is employed. For example, when the biological material 246 is to be used as a biological sensor in device 10, chambers 242 can be lower parts 29 of reaction chambers 12, and chambers 244 can be upper parts 31 thereof (see FIG. 2b). Thus, chamber 244 is preferably designed sizewise and shapewise compatible with chamber 242 so as to allow covering of chamber 242 with chamber 244. To facilitate the contact between chamber 242 and chamber 244, and the sealing of chamber 12 once chambers 242 and 244 are assembled together, an additional layer 243 may be placed between chambers 242 and 243. Additional layer 243 is preferably made of a rubbery material or any other material having sealing properties.

According to a preferred embodiment of the present invention biological material 246 is immobilized onto chamber 242. This can be done, for example, by encapsulating biological material 246 into a meltable membrane or a matrix made of, e.g., agar, alginate, poly-vinyl alcohol, sol-gel, carraginan and the like.

In a second step of the method, illustrated in FIG. 20b, chambers 242 and 244 are placed in a negative pressure environment 250, so as to dehydrate biological 10 material 246. According to a preferred embodiment of the present invention chamber 244 comprises a window 252, so that when chamber 244 partially covers chamber 242, evaporation of liquids is allowed through window 252.

In a third set of the method, illustrated in FIG. 20c, chamber 244 are positioned on chamber 242 in a manner such that chamber 242 is sealed. This can be done by pressing mechanical chamber 244 in the direction of chamber 242 until window 252 is completely sealed by walls 254 of chamber 242. According to a preferred embodiment of the present invention the sealing of window 252 is done in a manner such that fluid channel 248 is not obstructed. The sealing of chamber 242 is preferably executed while both chamber 242 and chamber 244 are in environment 250 so as to maintain negative pressure therein. Once chamber 242 is sealed by chamber 244 they can be removed from environment 250. However, the internal negative pressure of chambers 242 and 244 is preferably maintained, so as to prevent humidity and gasses (e.g., air, oxygen) from penetrating into chamber 12. The internal negative pressure is particularly useful when the present method is used for manufacturing device 10 for the purpose of future use. In such cases, it is desired to have the biological sensors in an inactive state while device 10 is not being used. One ordinarily skilled in the art would appreciate that as long as humidity and gasses are prevented from penetrating the chamber, the biological sensors remain inactive. Only once the sample fluid is transported through channel 248, the desired biological activity is initiated.

Thus, the present invention successfully provides a method of placing biological sensors in device 10, in a manner such that (i) negative pressure is maintain so that the biological sensors are in a dormant state until device 10 is operative; and (ii) the fluid channels are not obstructed so that when device 10 becomes operative, the sample fluid can be transported to the reaction chambers through the fluid channels as further detailed hereinabove. When the sample fluid is transported into device 10, the meltable membrane is melted thereby facilitating interaction between the biological sensor and the sample fluid. As a result of the interaction, the biological sensor produces a material generating the detectable signals. As stated, the produced material can be a fluorescent material so that optical signals are generated thereby.

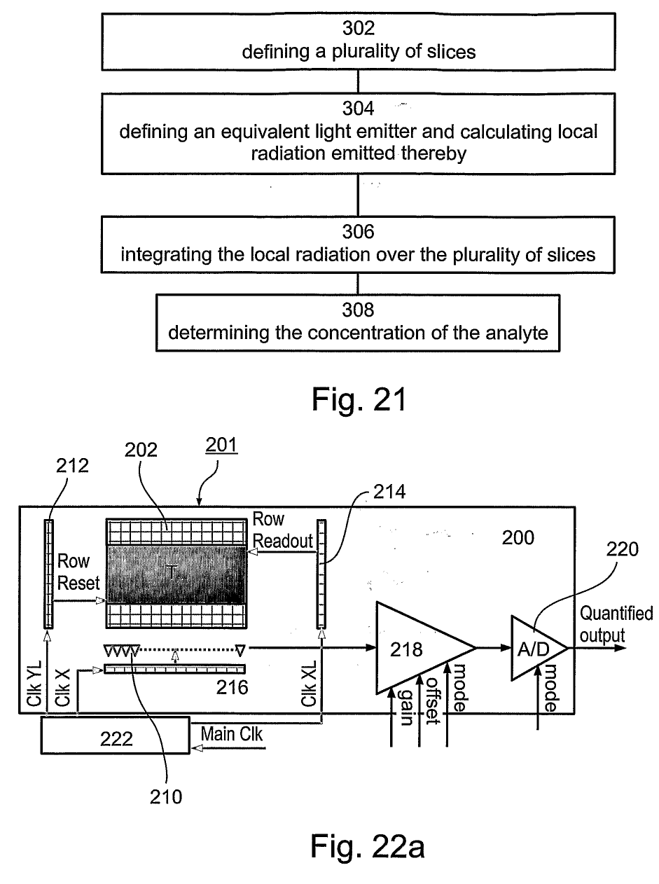

It is appreciated that when the sensors are biological sensors, each reaction chamber may contains many biological reporters, each located in a different location in the reaction chamber, hence contributes differently to the overall detected optical signal. The ability to accurately determine the concentration of the analyte therefore depends on a judicious calculation of the different contributions thereto. The present invention successfully provides a method of determining the concentration of the analyte in the fluid sample, from the optical signals generated by the fluorescent material. The method is based on a slicing technique and comprises the following method steps which are illustrated in the flowchart diagram of FIG. 21.

Hence, in a first step, designated by block 302, a plurality of slices is defined, where each slice includes at least one biological reporter. Preferably, the definition of slices is done for each of the reaction chamber of device 10. Ideally, each slice is two dimensional, however, although not excluded, such slices are hardly attainable. Thus, according to a preferred embodiment of the present invention the slices are defined such that the thickness of each slice is small, preferably about one third, more preferably one tenth, most preferably one hundredth or one thousandth of the depth of the reaction chamber. A representative example of calculating the thickness of a slice is given in the Examples section that follows (see Example 2).

In the second step of the method, designated by Block 304, all the biological reporters of a slice are represented as at least one equivalent light emitter, located at a predetermined location within the slice, for example, at the center of the slice. For each slice, the respective equivalent light emitter imitates all the biological reporters in the slice. In other words, the detected radiation is composed of a plurality of local radiation contributions, each emitted by one of the equivalent light emitters. The calculation of the local radiation contribution of each equivalent light emitter preferably comprises calculation of effective quantum efficiency preferably both for emission and for excitation, and at least one transmission coefficient corresponding to the excitation light and the light emitted by the equivalent light emitter.

In a third step of the method, designated by Block 306, the local radiation contribution is integrated over all or most of the slices of each reaction chamber so as to obtain an integrated radiation intensity. The integration in this step is preferably done along an axes perpendicular to the plane of each slice.

In the forth step of the method, designated by Block 308, the recorded optical signals and the integrated radiation intensity are used for determining the concentration of the analyte. This can be done by calculating an occupation area of the fluorescent material, in each reaction chamber. The occupation area is preferably defined as a projection of an occupation volume on a plane perpendicular to a direction of the excitation light. The occupation volume depends on the intensity of radiation emitted by fluorescent material. Thus, according to a preferred embodiment of the present invention the occupation volume is calculated by tracing the light rays emitted by the equivalent light emitters using optical geometry techniques, as further detailed in the Examples section that follows (see Example 2).

Additional objects, advantages and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non limiting fashion.

### Example 1

### CMOS Detector

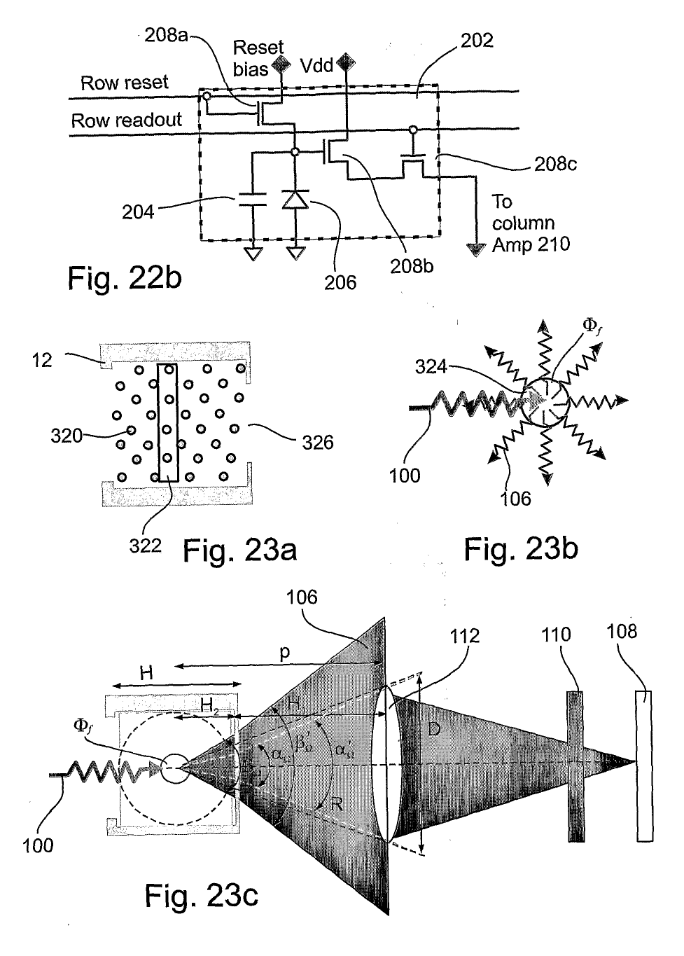

Reference is now made to FIG. 22a-b exemplifying an electronic diagram of a CMOS detector 200, which can be used as detector 108, according to a preferred embodiment of the present invention. CMOS detector 200 is known in the art and can be purchased, for example, from Fill Factory, Mechelen Belgium.

CMOS detector 200 comprises a matrix 201 of elementary units 202 referred to herein as pixels 202. FIG. 22b shows one pixel 202, which comprises a capacitor 204 which is pre-charged to a reset bias voltage, a photodiode 206, for discharging capacitor 204 in response to photons absorption and 3 MOS transistors, designated 208a, 208b and 208c, for resetting (transistor 208a), sensing (transistor 208b) and leading (transistor 208c) the signal to column amplifier 210.

CMOS detector 200 further comprising a left vertical shift register 212 and a right vertical shift register 214. Left register 212 serves a pointer to a row that is pre-charged to reset bias voltage (reset row operation) and right register 214 serves as a pointer to a row which is being read by column amplifier 210. The distance between two pointers determines the integration time, Tᵢₙₜ, which can be calculated as follows:
Tᵢₙₜ=N_{int-rows}·(T_{rd-px}·N_{X}+T_{blanking}), (EQ. 1)
where N_{int-rows} is the distance in rows between the readout and the reset row pointers, T_{rd-px} is the time for one pixel readout, N_{X} is the size of the window in X-direction and the T_{blanking} is the minimal required time between two successive row readouts. T_{rd-px} and T_{blanking} depend on signals generated in a clock unit 222.

CMOS detector 200 further comprises a column shift register 216 which selects the appropriate column amplifier 210. The signal from column amplifier 210 is transmitted through a pre-amplifier 218 to a digital converter 220.

Pre-amplifier 218 and converter 220 preferably operate in linear mode. The quantified output of CMOS detector 200 is proportional to the number of photons colliding on pixels 202, denoted Nₚₕₜ:

$${N}_{pht} ( {T}_{int} ) = C \frac{( QO ( {T}_{int} ) - {QO}_{DC\_e -} ( {T}_{int} ) ) / {2}^{bits}}{QE \cdot FF} , \qquad ( EQ . 2 )$$

where QO is a quantified output, bits is the quantization of converter 220, QE is a quantum efficiency of the CMOS detector 200, FF is a fill-factor, C is a maximal capacity of pixel in photo-electrons and QO_{DC}_{_{—}}ₑ₋ is a quantified output of an integrated dark current, I_{DC}.

The time dependence of QO_{DC}_{_{—}}ₑ₋ is given by:

$${QO}_{DC\_e -} ( T ) = \frac{{I}_{DC}}{C} T \cdot {2}^{bits} , \qquad ( EQ . 3 )$$

where the units of I_{DC}, C and T are such that the expression I_{DC}T/C is dimensionless. For example, I_{DC}, C and T can be measured in e⁻/s, e⁻ and s, respectively.

For the CMOS detector of the present example, a typical value of C, is 6×10⁴. The value of the dark current, I_{DC}, is about 1055 [e⁻/s], for a temperature of 21° C.

The required integration time is inversely proportional to the intensity of the signals. On the other hand, this maximal integration time is limited because of dark current saturation effect.

For weak signals, the maximal integration time is given by:

$${T}_{int - max - ws} = \frac{C}{{I}_{DC}} \cdot {F}_{DC} \qquad ( EQ . 4 )$$

where F_{DC} is a parameter defined by the following equation:
QO_{DC}_{_{—}}ₑ₋_{_{—}}ₘₐₓ=F_{DC}·2^{bits}. (EQ. 5)
A typical value of F_{DC} is about 0.5.

For strong signals, the maximal integration time is given by:

$${T}_{int - max - ss} = \frac{C}{{I}_{s} \cdot QE \cdot FF} , \qquad ( EQ . 6 )$$

where Iₛ is the strong signal current.

### Example 2

### Determination of Analyte Concentration Using the Detected Signals

Reference is now made to FIGS. 23a-c, which illustrate the radiation emitted by one reaction chamber of device 10.

FIG. 23a illustrates reaction chamber 12, a plurality of locations 320, where biological cells generating the florescent materials are located, and a slice 322 defined in accordance with a preferred embodiment of the present invention. Reaction chamber 12 has an aperture 326 through which optical signals 106 (not shown, see FIGS. 23b-c) exit. In the following calculations, slice 322 is represented as equivalent light emitter 324, shown in FIG. 23b. Equivalent light emitter 324 is a superposition of the all the light emitter in slice 322 and can be defined, for example, by integration or summation. Also shown is excitation light 100 and optical signal 106 emitted in a plurality of directions.

FIG. 23c illustrates the spreading of optical signal 106 through aperture 326 of the reaction chamber. The respective numerical aperture for optical signal 106, designated herein by β′_{Ω}, depends on the position of slice 322. Optical signal 106 is collimated by lens 112 prior to impingement of optical signal 106 on light detector 108. Being spaced apart from aperture 326 the corresponding numerical aperture of lens 112, designated herein by α′_{Ω}, is smaller than the numerical aperture of aperture 326, β′_{Ω}. Similarly to β′_{Ω}, α′_{Ω} also depends on the position of slice 322. Emission filter 110 is positioned in the light path of optical signal 106 so as to prevent rays of excitation light 100 from arriving to detector 108. Emission filter 110 preferably allows transmission of optical signal 106 substantially without loses.

The use of lens 112 is preferred, but optional. In an alternative embodiment in which lens 112 is not used, emission filter 110 and detector 108 are preferably positioned instead of lens 112, with no substantially change in the values of the β′_{Ω} and α′_{Ω}.

According to a preferred embodiment of the present invention several optical coefficients are calculated. A first optical coefficient is the emission quantum efficiency, denoted herein by EmQE, which is the ratio between the absorbed excitation radiation and the emitted radiation. A second optical coefficient is the transmission coefficient of emission filter 110 for excitation light 100, designated herein by T_{Fem4exc}. A third optical coefficient is the transmission coefficient of emission filter 110 for optical signal 106, designated herein by T_{Fem4ems}. A fourth optical parameter is the effective quantum efficiency of detector 108 for optical signal 106, designated herein by QEₑₘₛ, and a fifth optical parameter is the effective quantum efficiency of detector 108 for excitation light 100, designated QE_{exc}.

The value of the above five coefficients depends on the spectral characteristics of the optical components. More specifically, the following spectra are used for the calculations: (i) the spectrum of the light source, S_{L}; (ii) the spectrum of excitation filter S_{Fexc} (iv) The spectrum of emission filter 110, S_{Fem}; (v) the efficiency of the fluorescence excitation E_{fl}; (vi) the emission spectrum, Sₑₘₛ; and (vii) the Quantum Efficiency (QE) of light detector 108.

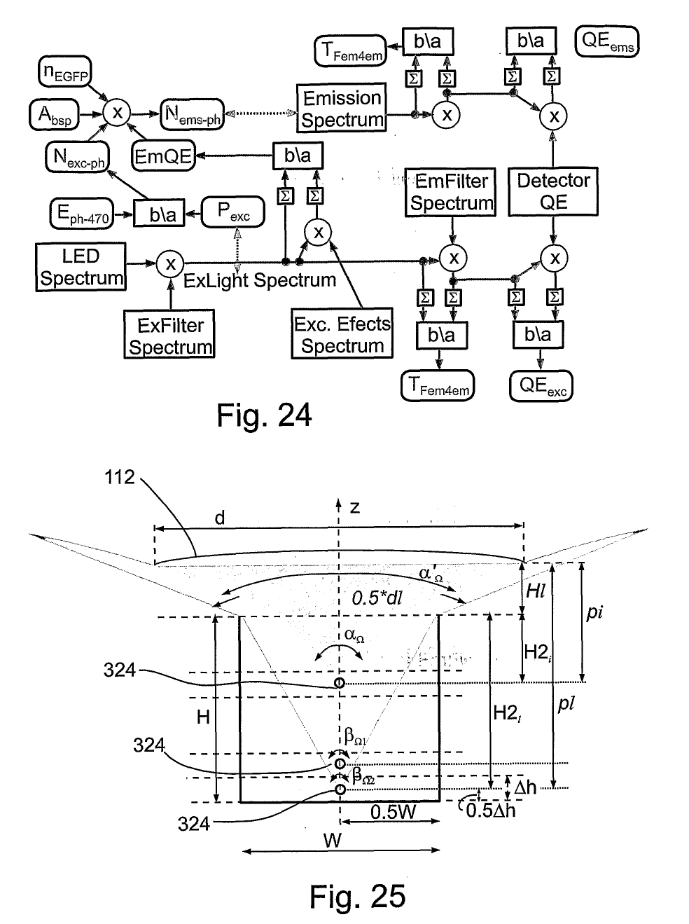

The calculation of the optical coefficient can be done using the following formulae:

$$\begin{matrix}EmQE = \frac{{\sum }_{λ} {S}_{L} {S}_{Fexc} {E}_{fl}}{{\sum }_{λ} {S}_{L} {S}_{Fexc}} , & ( EQ . 7 a ) \\ {T}_{Fem 4 exc} = \frac{{\sum }_{λ} {S}_{L} {S}_{Fexc} {S}_{Fems}}{{\sum }_{λ} {S}_{L} {S}_{Fexc}} , & ( EQ . 7 b ) \\ {T}_{Fem 4 ems} = \frac{{\sum }_{λ} {S}_{ems} {S}_{Fems}}{{\sum }_{λ} {S}_{ems}} , & ( EQ . 7 c ) \\ {QE}_{ems} = \frac{{\sum }_{λ} {S}_{ems} {S}_{Fems} QE}{{\sum }_{λ} {S}_{ems} {S}_{Fems}} , & ( EQ . 7 d ) \\ {QE}_{exc} = \frac{{\sum }_{λ} {S}_{L} {S}_{Fexc} {S}_{Fems} QE}{{\sum }_{λ} {S}_{L} {S}_{Fexc} {S}_{Fems}} , & ( EQ . 7 e )\end{matrix}$$

where A_{bsrp} is the ratio between the area of equivalent light emitter 324 to the area of the excitation beam, n_{GFP}, is the occupation of the fluorescent material, measured as a percentage of the area of equivalent light emitter 324, Nₑₘₛ₋ₚₕ is a number of the emission photons per second, N_{exc-ph} is a number of the excitation photons per second, Eₚₕ₋₄₇₀ is energy of single photon at the wavelength 470 nm and P_{exc} is the optical power. FIG. 24 is a schematic calculation diagram which can be implemented for the calculation of Equations 7a-7e.

According to a preferred embodiment of the present invention once the optical coefficients are calculated, a ray tracing procedure is employed.

FIG. 25 illustrates light propagation from equivalent light emitter 324 to lens 122. Light rays are redirected to the angle β′_{Ω}, according to Snell's law
n_{RC}·sin(β_{Ω}/2)=n·sin(β′_{Ω}/2) (EQ. 8)
where n_{RC} is the refraction index of the medium in reaction chamber 12 and n is the refraction index of the external medium.

Assuming that the there are B_{cube} biological reporters in a 1 mm³ cube, the number of biological reporters in one dimension B_{1D}, is the cubic root of B_{cube}. Thus, denoting the height of reaction chamber by H, the total number of slices is:
N_{layers}=B_{1D-1 mm}·(H/1 mm), (EQ. 9)
Thus, each slice has a thickness of:
Δh=H/N_{layers}, (EQ. 10)
and is centered at position H₂ᵢ, where:
H₂ᵢ=H−Δh·(i−0.5). (EQ. 11)

The scattering angle of optical signal from the ith slice is given by:
β_{Ωi}=2·tg⁻¹(W/2H₂ᵢ), (EQ. 12)
where W is the diameter of aperture 326.

Using Equations 8 and 12 one can calculate the numerical aperture, β′_{Ω}:
β′₁₀₆=2·sin⁻¹((n_{RC}/n)·sin(tg⁻¹(W/2H₂ᵢ))). (EQ. 13)

The numerical aperture of the lens for the ith slice is given by:
α_{Ωi}=2·tg⁻¹(d1/2H2ᵢ), (EQ. 14)
where H₂ᵢ is the distance between the center of the ith slice and output aperture and d1 is twice the distance between the optical axis of the lens and the emitted light ray (see FIG. 25), which can be numerically calculated, for example, by an iterative procedure.

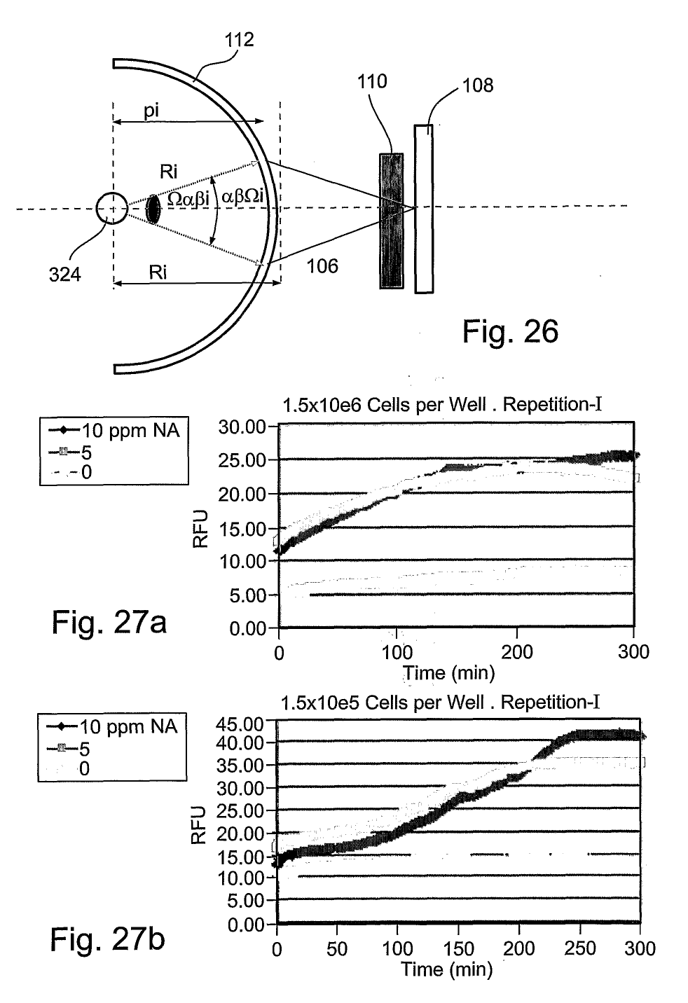

FIG. 26 illustrates the scattering solid angle of the emitted light rays. Hence, defining the range αβ_{Ωi} as min(α_{Ωi}, β_{Ωi}), light rays emitted from the ith slice at an angle within range αβ_{Ωi}, are redirected to detector 108.

Generally, rays 106 are scattered uniformly to a solid angle of 4π. The fraction of emission energy impinging on detector 108 is therefore:

$${F}_{GFP} = \frac{{Ω}_{α β i}}{4 π} , \qquad ( EQ . 14 )$$

where Ω_{αβi} is the solid angle corresponding to range αβ_{Ωi} and is given by:
Ω_{αβi}=2π(1−cos(0.5=αβ_{Ωi})). (EQ. 15)

Following is a description of a calculation of the absorption of the excitation light and the corresponding emission of optical signals 106.

A photon having wavelength λ carries energy which equals:

$${E}_{ph} ( λ ) = \frac{h \cdot c}{λ} . \qquad ( EQ . 16 )$$

For example, when the excitation wavelength is 470 nm, the energy carried by one excitation photon is 3.810⁻¹⁹ J. Assuming that the excitation light is transmitted by an optical fiber, photon flux (the number of photon per unit time) is given by:

$${I}_{exc} = \frac{{P}_{fiber}}{{E}_{ph} ( {λ}_{exc} )} , \qquad ( EQ . 17 )$$

where P_{fiber} is the optical power of the optical fiber which can be measured, for example, using an optical power meter positioned on the output of the optical fiber.

The amount of optical signals generated by the biological material is proportional to the projection area of the fluorescent material on a plane perpendicular to the direction defined by detector 108 and slice 324.

For a d_{b}×d_{b}×d_{b} cube, the maximum absorption by the fluorescent material of the ith slice is:

$${A}_{GFP - max - i} = \frac{{d}_{b}^{2} \cdot {B}_{layer}}{π \cdot {( W / 2 )}^{2}} , \qquad ( EQ . 18 )$$

where B_{layer} is the number biological reporters in ith slice:

$${B}_{layer} = \frac{{B}_{RC}}{{N}_{layers}} . \qquad ( EQ . 19 )$$

The maximal absorption occurs for when the biological material is completely saturated by the fluorescent material. The efficiency of the biochemical reaction is proportional to the percentage of the biosensor saturation by the GFP molecules. The percentage of the saturation in layer i is signed as n_{GFP-i}.

The light absorption in the ith layer is given by:
A_{GFP-i}=A_{GFP-max-i}·n_{GFP}, (EQ. 20)
where A_{GFP-max-i} is the maximal absorption.

The value of the n_{GFP} can be between zero and unity inclusive. n_{GFP}=0 means that is that no fluorescent material was generated, while n_{GFP}=1 means that the entire biological cell is saturated by fluorescent material and the absorption equals A_{GFP-max-i}.

Assuming that the dominant attenuation of the excitation light is due to the fluid in the reaction chamber, the absorbed excitation light in the ith slice is given by:
I_{exc-i}=I_{exc-i-1}·T_{exc}(Δh)=I_{exc}·T_{exc}ⁱ⁻¹(Δh). (EQ. 21)

The maximal intensity of the emitted optical signal, scattered at a solid angle of 4π, can be written as:
Iₑₘₛ₋ₘₐₓ₋ᵢ=A_{GFP-max-i}·I_{exc-i}·EmQE, (EQ. 22)
and the optical signal intensity as function of the bio-chemical reaction efficiency is therefore given by:
Iₑₘₛ₋ᵢ=Iₑₘₛ₋ₘₐₓ₋ᵢ·n_{GFP}. (EQ. 23)

Integrating over all slices one thus obtain a relation between the detected signal and n_{GFP}. One ordinarily skilled in the art would appreciate that from the value of n_{GFP} the concentration of the analyte can be obtained, for example, using a simple calibration curve.

### Example 3

### Sensitivity Calculation

As stated, the emission intensity is proportional to the biochemical reaction percentage expressed by the n_{GFP} parameter. In the present example, a sensitivity calculation is performed using a signal uncertainty parameter, which is proportional to the unfiltered excitation intensity detected by the light detector:
I_{unc-px}=Un2B·I_{exc-px}·QE_{exc}, (EQ. 24)
where I_{unc-px} is the signal uncertainty parameter, Un2B is the ratio between the uncertainty to the background radiation, Iₑₘₛ₋ₚₓ is the unfiltered excitation intensity as detected by the light detector and QE_{exc} is, as stated, the effective quantum efficiency of the detector for excitation light. A typical value for Un2B is about 0.5.

The minimal sensitivity is preferably defined such that the sensed optical signal is at least S2Un times stronger that the signal uncertainty, where S2Un is the ratio between the signal to the uncertainty:
Iₑₘₛ₋ₚₓ·QEₑₘₛ≧S2Un·I_{unc-px}, (EQ. 25)
where Iₑₘₛ₋ₚₓ is the emission intensity as detected by the light detector, and QE_{exc} is the effective quantum efficiency of the detector for emission light. The emission intensity from the biological sensor is proportional to the n_{GFP}, thus:
R_{ems/exc}=R_{ems/exc-max}·n_{GFP}
Iₑₘₛ₋ₚₓ=Iₑₘₛ₋ₘₐₓ₋ₚₓ·n_{GFP}, (EQ.26)
where, R_{ems/ex} is the ratio between emission and excitation, R_{ems/ex-max} is the maximal ratio between emission and excitation and Iₑₘₛ₋ₚₓ is the maximal emission intensity which can be detected.

Combining the Equations 24-26 one has:

$$\frac{{I}_{ems - max - px} \cdot {QE}_{ems}}{{I}_{exc - px} \cdot {QE}_{exc}} \cdot {n}_{GFP} \geq S 2 Un \cdot Un 2 B , \qquad ( EQ . 27 )$$

Denoting:

$${R}_{ems / exc - max} = \frac{{I}_{ems - max - px} \cdot {QE}_{ems}}{{I}_{exc - px} \cdot {QE}_{exc}} , \qquad ( EQ . 28 )$$

Equation 27 can be written as:
R_{ems/exc-max}·n_{GFP}≧S2Un·Un2B. (EQ. 29)

The constant R_{ems/exc-max} depends on the optical setup, and can be determined, experimentally, or by geometrical optic calculations. From Equation 29 it follows that the minimal optical system sensitivity to the bio-chemical reaction is:

$${n}_{GFP - min} = \frac{S 2 Un \cdot Un 2 B}{{R}_{ems / exc - max}} . \qquad ( EQ . 30 )$$

### Example 4

### Biological Signal Quantification

In this example, the biological signal quantification is demonstrated for a system in which excitation energy is transmitted using external optical fibers and the sensors are E. coli bacteria, capable of producing GFP molecules when interacting with a toxic material. The bacteria were placed in the reaction chamber in the presence of a Luria Bertani nutrition medium.

The detected signal, QO_{int-sig}, is a sum of the following contributions (i) unfiltered excitation intensity, I_{exc-Fem4exc}; (ii) intensity of emission generated by nutrient medium, I_{LB}; (iii) intensity of light emitted by the GFP molecules, I_{GFP}; and (iv) dark current contribution of the light detector, I_{DC}.

The overall emission intensity that reaches the pixels of the detector is given by:

$${I}_{ems - mlens - det} = {\sum }_{i = 1}^{i = {N}_{layers}} ( \begin{matrix}{A}_{GFP - max} \cdot {I}_{exc - i} \cdot {n}_{GFP} \cdot \\ {T}_{ems} ( {H}_{2 i} ) \cdot \frac{{Ω}_{αβ i}}{4 π} \cdot {T}_{mlens} \cdot {T}_{Fems4ems}\end{matrix} ) \qquad ( EQ . 31 )$$

For an excitation wavelength of 470 nm, the excitation at the ith slice is:

$${I}_{exc - i} = ( \frac{{P}_{fiber}}{{E}_{ph} ( 470 n m )} ) \cdot {T}_{exc}^{i - 1} ( Δ h ) = {P}_{fiber} \cdot ( \frac{{T}_{exc}^{i - 1} ( Δ h )}{{E}_{p h} ( 470 n m )} ) . \qquad ( EQ . 32 )$$

The light intensity, emitted by the GFP molecules is therefore given by:

$$\begin{matrix} {I}_{ems - mlens - det} = \\ = {\sum }_{i = 1}^{i = {N}_{layers}} ( \begin{matrix}{A}_{GFP - max} \cdot {P}_{fiber} \cdot ( \frac{{T}_{exc}^{i - 1} ( Δ h )}{{E}_{p h} ( 470 n m )} ) \cdot {n}_{GFP} \cdot \\ {T}_{ems} ( {H}_{2 i} ) \cdot \frac{{Ω}_{αβ i}}{4 π} \cdot {T}_{mlens} \cdot {T}_{Fems 4 ems}\end{matrix} ) = \\ = {n}_{GFP} \cdot {P}_{fiber} \cdot \underbrace{{\sum }_{i = 1}^{i = {N}_{layers}} ( \begin{matrix}{A}_{GFP - max} \cdot ( \frac{{T}_{exc}^{i - 1} ( Δ h )}{{E}_{p h} ( 470 n m )} ) \cdot {T}_{ems} ( {H}_{2 i} ) \cdot \\ \frac{{Ω}_{αβ i}}{4 π} \cdot {T}_{mlens} \cdot {T}_{Fems 4 ems}\end{matrix} )}_{{K}_{bs - exc - ems - optics - mlens}}\end{matrix} \qquad ( Eq . 33 )$$

where K_{bs-exc-ems-optics-mlens} is a constant coefficient depending on the geometry of the reaction chamber, the concentration of bacteria, the optical characteristics of the optical components (filters and lenses) and the photon energy. The emission intensity which achieves the light detector is given by:
Iₑₘₛ₋ₘₗₑₙₛ−det=n_{GFP}·P_{fiber}·K_{bs-exc-ems-optics-mlens}. (EQ. 34)

Denoting the area onto which the optical signal is spread on the detector by S_{ems-det}, and the area of one pixel by Sₚₓ, the emission intensity projected on one pixel is:
Iₑₘₛ₋ₘₗₑₙₛ₋ₚₓ=n_{GFP}·P_{fiber}·K_{bs-exc-ems-optics-mlens}·(Sₚₓ/S_{ems-det})=n_{GFP}·P_{fiber}·K_{bs-px} (EQ. 35)
where, K_{bs-px} is a numerical coefficient which can be written as:

$${K}_{bs - px} = \frac{{S}_{px}}{{S}_{ems - det}} \cdot {\sum }_{i = 1}^{i = {N}_{layers}} ( \begin{matrix}{A}_{GFP - max} \cdot ( \frac{{T}_{exc}^{i - 1} ( Δ h )}{{E}_{p h} ( 470 n m )} ) \cdot \\ {T}_{ems} ( {H}_{2 i} ) \cdot \frac{{Ω}_{αβ i}}{4 π} \cdot {T}_{mlens} \cdot {T}_{Fems 4 ems}\end{matrix} ) \qquad ( EQ . 36 )$$

The relation between the quantified output (QO) of the light detector and the number of photons is given by:

$${N}_{pht} ( {T}_{int} ) = C \frac{( QO ( {T}_{int} ) - {QO}_{D C\_e -} ( {T}_{int} ) ) / {2}^{bits}}{QE \cdot FF} , \qquad ( EQ . 37 )$$

where the quantified output due to dark current, QO_{DC}, can be measured by switching off the excitation light source.

Denoting the quantified GFP molecules emission by QI_{GFP}, the occupation of the fluorescent material can be written as:

$${n}_{GFP} = {QI}_{GFP} \cdot ( \frac{1}{{T}_{int} \cdot {P}_{fiber}} ) \cdot ( \frac{1}{{K}_{bs - px}} \cdot \frac{( 6 e 4 / {2}^{bits} )}{QE \cdot FF} ) \qquad ( EQ . 38 )$$

The separation of QI_{GFP} from QO_{int-sig}, can be done by logical division of the reaction chambers, in which in a first reaction chamber only the nutrition medium is placed (without the bacteria), hence generates only the contribution of I_{exc-Fem4exc}, I_{LB} and, I_{DC}. In a second reaction chamber both the nutrition medium and the bacteria are present hence generates, once interacting with the fluid sample, all the aforementioned contributions (i)-(iv).

The quantified emission intensity of the GFP molecules is thus obtained by subtraction:
QI_{GFP}=QO₂−QO₁, (EQ. 39)
where QO₁ and QO₁ are, respectively, the quantified output received from the first reaction chamber (without the bacteria) and the second reaction chamber (with the bacteria).

### Example 5

### Experimental

A prototype system was built according to the teaching of preferred embodiments of the invention described above. The prototype system included a detecting device with 12 reaction chambers and a plurality of micro-pumps were employed (see FIG. 3a), and a CMOS light detector (see Example 1). The sensors were E. coli-REC-A bacteria, referred to in this Example as biosensors.

The reaction chamber of the device were used as follows: (2 repetitions)×(2 biosensors E. coli—rec-A quantities: 1.5×10⁶, 1.5×10⁵ [bacteria/1 uL])×(3 Nalidixic Acid concentrations: 0, 5, 10 parts per million. The experiment duration was 5 hours.

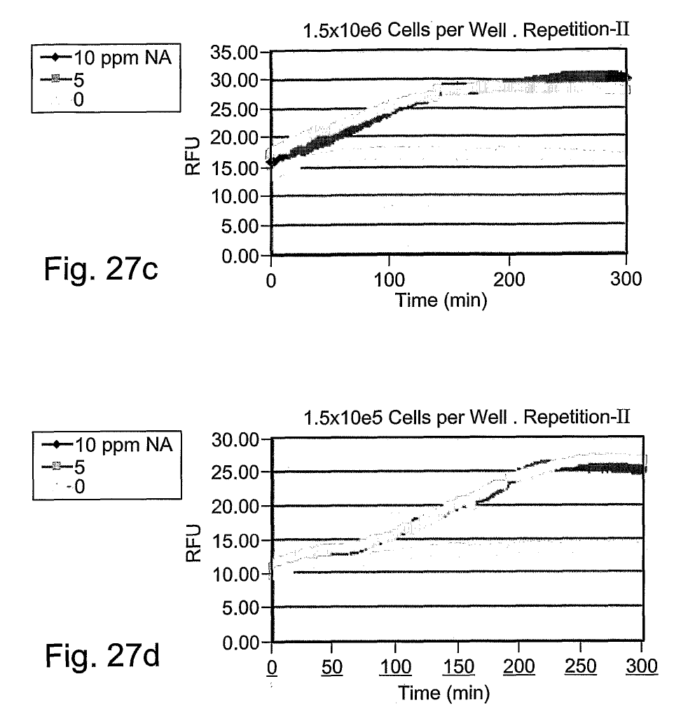

FIGS. 27a-d show the detected optical signal as a function of time. FIGS. 27a and 27c show the detected optical signal for a concentration of 1.5×10⁶ cells per reaction chamber (first and second repetitions, respectively), and FIGS. 27b and 27d show the detected optical signal for a concentration of 1.5×10⁵ cells per reaction chamber (first and second repetitions, respectively).

1-242. (canceled)

243. A device for detecting a presence of an analyte in a sample comprising a device body configured with at least one reaction chamber configured for containing a sensor capable of producing a detectable signal when exposed to the analyte in the sample, said at least one reaction chamber being in fluid communication with at least one sample port and at least one actuator port via a first set of microfluidic channels arranged such that application of a negative pressure to said at least one actuator port delivers fluid placed in said at least one sample port to said at least one reaction chamber.

244. A system for detecting at least one analyte present in a sample, the system comprising:
a detecting device having a plurality of reaction chambers and a plurality of channels interconnecting at least a portion of said plurality of reaction chambers, wherein at least a portion of said plurality of reaction chambers comprises a sensor, capable of generating a detectable optical signal when exposed to the at least one analyte;
a planar light detector capable of receiving optical signals from said detecting device and providing an image of sensors generating said optical signals.

245. The system of claim 244, further comprising a data processor, supplemented by an algorithm for receiving image information from said planar light detector and determining presence of the at least one analyte.

246. The system of claim 244, further comprising a control unit for sending control signals to said detecting device.

247. The system of claim 244, further comprising a temperature control unit for controlling a temperature of said detecting device and/or said planar light detector.

248. The system of claim 245, wherein said algorithm is capable of determining concentration of the at least one analyte.

249. The system of claim 244, wherein said plurality of reaction chambers are configured so as to enable sustaining a negative pressure environment within said plurality of reaction chambers.

250. The system of claim 244, wherein at least a portion of said plurality of reaction chambers are sequentially interconnected via at least a portion of said channels.

251. The system of claim 244, wherein a body of said detecting device is capable of allowing transmission of light having a predetermined wavelength therethrough.

252. The system of claim 244, wherein said sensor is a biological sensor.

253. The system of claim 244, wherein said biological sensors is capable of producing a bioluminescent material.

254. The system of claim 244, wherein said biological sensors is capable of producing a phosphorescent material.

255. The system of claim 244, wherein said biological sensor is capable producing a fluorescent material.

256. The system of claim 244, wherein said planar light detector comprises a matrix having a plurality of addressable elementary units, each being capable of converting said optical signal into an electrical signal.

257. The system of claim 244, wherein said planar light detector is selected from the group consisting of a CCD camera and a CMOS detector.

258. The system of claim 244, further comprising a light source for emitting excitation light so as to excite said sensor to thereby emit said optical signal.

259. The system of claim 258, further comprising at least one selective filter positioned between said detecting device and said planar light detector, said at least one selective filter being capable of transmitting said optical signals and preventing transmission of said excitation light.

260. The system of claim 258, further comprising a plurality of optical fibers for guiding said excitation light into said detecting device.

261. The system of claim 244, further comprising an optical focusing device for focusing said optical signal on said planar light detector.

262. The system of claim 244, further comprising a transport mechanism for actuating transport of a sample fluid in said plurality of fluid channels, thereby to fill said plurality of reaction chambers with said sample fluid.

263. The system of claim 262, wherein said transport mechanism comprises a pumping device, capable of generating a negative pressure in said plurality of reaction chambers and said plurality of fluid channels.

264. The system of claim 263, wherein said pumping device comprises a plurality of micro-pumps

265. A device for detecting at least one analyte present in a sample, the device comprising at least one array of reaction chambers, each array having a plurality of reaction chambers, sequentially interconnected by a plurality of fluid channels, in a manner such that each reaction chamber is in direct fluid communication with at least two other reaction chambers, whereby a first reaction chamber of said at least two other reaction chambers serves as a fluid source and a second reaction chamber of said at least two other reaction chambers serves as a fluid sink, wherein each reaction chamber is designed for containing a sensor capable of generating a detectable signal when exposed to the at least one analyte.

266. A device for detecting at least one analyte present in a sample, the device comprising a substrate configured with:
(a) a plurality of chambers for holding a fluorescent sensor and incubating reaction between said fluorescent sensor and the at least one analyte;
(b) a plurality of fluid channels interconnecting at least a portion of said plurality of chambers; and
(c) a plurality of waveguides designed and constructed to distribute excitation light among said plurality of chambers in a manner such that impingement of said excitation light on said fluorescent sensor is maximized and impingement of said excitation light on a surface of said substrate is minimized.

267. An apparatus for imaging a pattern of optical signals received from a fluorescent material arranged in a plurality of predetermined locations, the apparatus comprising:
(a) a planar light detector engaging a first plane;
(b) a optical element engaging a second plane substantially parallel to said first plane;
(c) a Light source interposed between said first and said second planes, said light source capable of generating excitation light in a direction other than a direction of said planar light detector;
said optical element and said planar light detector being designed an constructed such that said excitation light is collimated by said optical element and impinges on at least a portion of said plurality of predetermined locations, and emission light, emitted by said fluorescent material in response to said excitation light, is focused by said optical element and impinges on said planar light detector, to form the pattern of the optical signal thereupon.

268. A method of determining concentration of an analyte from optical signals recorded of a reaction chamber in response to excitation light, the reaction chamber containing a plurality of biological sensors producing a fluorescent material when exposed to the analyte, the method comprising:
(a) defining a plurality of slices, each slice having at least one biological reporter;
(b) for each slice, representing said at least one biological reporter as at least one equivalent light emitter, located at a predetermined location within said slice, and calculating local radiation contribution emitted by said at least one equivalent light emitter; and
(c) integrating said local radiation contribution over said plurality of slices so as to obtain an integrated radiation intensity; and
(d) using the recorded optical signals and said integrated radiation intensity for determining the concentration of the analyte.

269. A method of detecting analytes in a sample fluid, comprising:
(a) providing a device having a plurality of reaction chambers and a plurality of channels, interconnecting at least a portion of said plurality of reaction chambers, wherein each one of said plurality of reaction chambers comprises a biological sensor, capable of generating a detectable signal when exposed to the at least one analyte;
(b) filling at least a first portion of said plurality of reaction chambers with the sample fluid;
(c) generating a condition for said biological sensor to generate said detectable signal; and
(d) detecting said detectable signal thereby detecting the analytes in the sample fluid.
